(19) United States
(12) Patent Application Publication
(10) Pub. No.: US 2026/0026529 A1
(43) Pub. Date: Jan. 29, 2026

(54) MATERIALS AND METHODS FOR THE MANUFACTURE OF AN ANIMAL FEED

(71) Applicant: In the Bowl Animal Health, Inc., Nashville, TN (US)

(72) Inventors: Kevin E. Willard, Knightstown, IN (US); Joseph R. Winkle, Carmel, IN (US); Scott Holmstrom, Fishers, IN (US); Robin S. Readnour, Nashville, TN (US)

(21) Appl. No.: 19/342,913

(22) Filed: Sep. 29, 2025

Related U.S. Application Data
(63) Continuation of application No. PCT/US2024/022531, filed on Apr. 1, 2024.
(60) Provisional application No. 63/493,741, filed on Apr. 1, 2023.
(60) Provisional application No. 63/571,839, filed on Mar. 29, 2024.

(51) Int. Cl. A23K 40/30; A23K 10/20; A23K 10/30; A23K 20/158; A61K 31/155; A61K 31/195; A61K 31/365; A61K 31/4184; A61K 31/42; A61K 31/422; A61K 31/495; A61K 31/4985; A61K 31/506; A61K 31/7048; A61K 38/13

(52) U.S. Cl. CPC A23K 40/30; A23K 10/20; A23K 10/30; A23K 20/158; A61K 31/155; A61K 31/195; A61K 31/365; A61K 31/4184; A61K 31/42; A61K 31/422; A61K 31/495; A61K 31/4985; A61K 31/506; A61K 31/7048; A61K 38/13

(57) ABSTRACT

Methods of manufacturing animal feed that include one or more active ingredients added to the feed post extrusion. The feeds can be dry or semi-dry kibble coated with an edible mixture that includes at least one active ingredient.

### PRIORITY CLAIM

This application claims priority to PCT/US2024/022531, filed on Apr. 1, 2024, which claims the benefit of U.S. Provisional Patent Application Nos. 63/571,839 filed on Mar. 29, 2024, and 63/493,741, filed on Apr. 1, 2023, the disclosures of which are hereby incorporated in their entirety herein by reference.

### FIELD OF THE INVENTION

This disclosure pertains to animal feeds, and materials and methods of making animal feeds.

### BACKGROUND

Administering medications to animals, especially pets, is an on-going challenge for people who keep pets that require medication. Specific challenges include persuading pets to ingest medications and the tendency of some owners to forget to administer medications to pets on the prescribed regimen. Some aspects of this disclosure address these and other challenges facing pet owners administering medications, functional feed ingredients, vitamins, minerals, enzymes, prebiotics and probiotics to pets by providing active kibble and methods of making the same.

### SUMMARY

Some aspects of this disclosure include methods of making animal feeds comprising the steps of mixing an active with a carrier to apply to an extruded kibble, applying the mixture to a kibble, and incorporating the mixture into the kibble to create a substantially homogenous activated feed.
Embodiment 1: A method of making an animal feed, including:
a) mixing at least one edible carrier to form a mixture, wherein the mixture optionally includes at least one surface-active agent;
b) maintaining the mixture at a temperature greater than 40.0° C.;
c) adding at least one active ingredient to the mixture to form an admixture; and
d) maintaining the admixture at a temperature of greater than 40.0° C.
Embodiment 2: The method according to embodiment 1, wherein the edible carrier, is selected from the group consisting of liquids and solids.
Embodiment 3: The method according to embodiments 1-2, wherein the admixture is at least one of the following: a solution; a suspension; a reverse emulsion; an emulsion; a powder; granules; and crystals.
Embodiment 4: The method according to embodiments 1-3, further including:
e) providing a dry or semi-dry kibble; and
f) applying at least a portion of the admixture to at least a portion of the kibble to form an active kibble.
Embodiment 5: The method according to embodiment 4, further including:
g) adding at least one additive to the active kibble.
Embodiment 6: The method according to embodiment 5, wherein the additive is selected from the group consisting of: palatants, bactericides, fungicides, and edible food colorings.
Embodiment 7: The method according to embodiments 4-6, further including:
h) tumbling the active kibble to create a substantially homogenous batch of active kibble.
Embodiment 8: The method according to embodiments 4-7, further including repeating one or more of steps f), g) and/or h).
Embodiment 9: The method according to embodiments 1-8, wherein the edible carrier is a synthetic or naturally occurring edible fat or oil.
Embodiment 10: The method according to embodiment 9, wherein the naturally occurring edible fat or oil is an animal fat selected from the group consisting of: chicken fat, turkey fat, duck fat, fish oil, tallow, sheep fat, and pork fat, or wherein the naturally occurring edible fat includes a vegetable fat or oil.
Embodiment 11: The method according to embodiments 1-8, wherein the edible carrier is a solid diluent.
Embodiment 12: The method according to embodiment 11, wherein the solid diluent is a powder, granules or crystals.
Embodiment 13: The method according to embodiments 11-12, wherein the solid diluent includes an anti-caking agent selected from the group consisting of aluminum calcium silicate, attapulgite clay, calcium silicate, calcium stearate, diatomaceous earth, magnesium silicate, sodium aluminosilicate, hydrated sodium calcium aluminosilicate, tricalcium silicate, hydrophobic silica, iron ammonium citrate, kaolin, montmorillonite clays, sodium silico aluminate and yellow prussiate of soda.
Embodiment 14: The method according to embodiments 1-13, wherein the solid diluent is selected from the group consisting of: a grain, a vegetable protein, an animal protein, and a sugar.
Embodiment 15: The method according to embodiments 1-14, wherein the surface-active agent is amphiphilic.
Embodiment 16: The method according to embodiments 1-15, wherein the surface-active agent is a surfactant.
Embodiment 17: The method according to embodiments 1-16, wherein the surface-active agent is a dispersant.
Embodiment 18: The method according to embodiments 1-17, wherein the admixture is one or more of the following: a solution; an emulsion; a reverse emulsion/vesicle; and a suspension.
Embodiment 19: The method according to embodiments 1-18, wherein the admixture further includes at least one palatant.
Embodiment 20: The method according to embodiments 1-19, wherein the admixture further includes at least one edible coloring agent.
Embodiment 21: The method according to embodiments 2-20, wherein at least a portion of the kibble is hydrophobic.
Embodiment 22: The method according to embodiments 2-21, wherein at least a portion of the kibble is hydrophilic.
Embodiment 23: The method according to embodiments 2-22, wherein at least a portion of the kibble is porous.
Embodiment 24: The method according to embodiments 2-23, further including treating the kibble to increase its affinity for the admixture.
Embodiment 25: The method according to embodiments 1-24, wherein the active ingredient is one or more compounds selected from the group consisting of: an anthelmintic, an acaricide, a miticide, an insecticide, an analgesic, an antibiotic and a metabolic regulator.
Embodiment 26: The method according to embodiments 1-25, wherein the active ingredient is mivorilaner.
Embodiment 27: The method according to embodiments 1-26, wherein the surface-active agent is lecithin.
Embodiment 28: The method according to embodiments 1-27, wherein the edible carrier is chicken fat.
Embodiment 29: The method according to embodiments 1-28, wherein the admixture is maintained at a temperature range between 40.00 C and 65.00 C, inclusive.
Embodiment 30: The method according to embodiments 1-29, further including heating the edible carrier to greater than 40.00 C before it is mixed with the surface-active agent and/or the active ingredient.
Embodiment 31: The method according to embodiments 1-30, wherein the admixture includes between about 0.5 wt. % to about 10 wt. % of lecithin.
Embodiment 32: The method according to embodiments 1-30, wherein the admixture includes between about 1.0 wt. % to about 6.0 wt. % of lecithin.
Embodiment 33: The method according to embodiments 1-30, wherein the admixture includes between about 2.0 wt. %, to about 5.0 wt. % of lecithin.
Embodiment 34: The method according to embodiments 1-30, wherein the admixture includes between about 3.5 wt. % to about 4.5 wt. % of lecithin.
Embodiment 35: The method according to embodiments 1-34, wherein the admixture includes between about 90.0 wt. % to about 99.5 wt. % edible fat.
Embodiment 36: The method according to embodiments 1-34, wherein the admixture includes between about 94.0 wt. % to about 99.0 wt. % edible fat.
Embodiment 37: The method according to embodiments 1-34, wherein the admixture includes between about 95.0 wt. % to about 98.0 wt. % edible fat.
Embodiment 38: The method according to embodiments 1-34, wherein the admixture includes between about 95.5 wt. % to about 96.5 wt. % edible fat.
Embodiment 39: The method according to embodiments 35-38, wherein the edible fat is chicken fat.
Embodiment 40: The method according to embodiments 1-39, wherein the active ingredient is mivorilaner.
Embodiment 41: The method according to embodiment 40, wherein the active kibble includes between about 30 mg of mivorilaner/kg of active kibble to about 350 mg of mivorilaner/kg of active kibble.
Embodiment 42: The method according to embodiment 40, wherein the active kibble includes between about 50 mg of mivorilaner/kg of active kibble to about 250 mg of mivorilaner/kg of active kibble.
Embodiment 43: The method according to embodiment 40, wherein the active kibble includes between about 60 mg of mivorilaner/kg of active kibble to about 200 mg of mivorilaner/kg of active kibble.
Embodiment 44: The method according to embodiments 1-39, wherein the active ingredient is moxidectin.
Embodiment 45: The method according to embodiment 44, wherein the active kibble includes between about 0.05 mg of moxidectin/kg of active kibble to about 1.0 mg of moxidectin/kg of active kibble.
Embodiment 46: The method according to embodiment 44, wherein the active kibble includes between about 0.08 mg of moxidectin/kg of active kibble to about 0.6 mg of moxidectin/kg of active kibble.
Embodiment 47: The method according to embodiment 44, wherein the active kibble includes about 0.147 mg of moxidectin/kg of active kibble.
Embodiment 48: The method according to embodiments 1-39, wherein the active ingredient is cyclosporin.
Embodiment 49: The method according to embodiment 48, wherein the active kibble includes between about 50 mg of cyclosporin/kg of active kibble to about 3,000 mg of cyclosporin/kg of active kibble.
Embodiment 50: The method according to embodiment 48, wherein the active kibble includes between about 100 mg of cyclosporin/kg of active kibble to about 2,500 mg of cyclosporin/kg of active kibble.
Embodiment 51: The method according to embodiment 48, wherein the active kibble includes between about 300 mg of cyclosporin/kg of active kibble to about 2000 mg of cyclosporin/kg of active kibble.
Embodiment 52: The method according to embodiments 1-39, wherein the active ingredient is fenbendazole.
Embodiment 53: The method according to embodiment 52, wherein the active kibble includes between about 30 mg of fenbendazole/kg of active kibble to about 500 mg of fenbendazole/kg of active kibble.
Embodiment 54: The method according to embodiment 52, wherein the active kibble includes between about 50 mg of fenbendazole/kg of active kibble to about 400 mg of fenbendazole/kg of active kibble.
Embodiment 55: The method according to embodiment 52, wherein the active kibble includes between about 60 mg of fenbendazole/kg of active kibble to about 300 mg of fenbendazole/kg of active kibble.
Embodiment 56: The method according to embodiments 1-39, wherein the active ingredient is gabapentin.
Embodiment 57: The method according to embodiment 56, wherein the active kibble includes between about 60 mg of gabapentin/kg of active kibble to about 5,000 mg of gabapentin/kg of active kibble.
Embodiment 58: The method according to embodiment 56, wherein the active kibble includes between about 150 mg of gabapentin/kg of active kibble to about 4,000 mg of gabapentin/kg of active kibble.
Embodiment 59: The method according to embodiment 56, wherein the active kibble includes between about 300 mg of gabapentin/kg of active kibble to about 3,000 mg of gabapentin/kg of active kibble.
Embodiment 60: The method according to embodiments 1-39, wherein the active ingredient is metformin.
Embodiment 61: The method according to embodiment 60, wherein the active kibble includes between about 250 mg of metformin/kg of active kibble to about 10,000 mg of metformin/kg of active kibble.
Embodiment 62: The method according to embodiment 60, wherein the active kibble includes between about 500 mg of metformin/kg of active kibble to about 7,000 mg of metformin/kg of active kibble.
Embodiment 63: The method according to embodiment 60, wherein the active kibble includes between about 1,500 mg metformin/kg of active kibble to about 5,000 mg of metformin/kg of active kibble.
Embodiment 64: The method according to embodiments 1-39, wherein the active ingredient is a combination of mivorilaner, moxidectin and fenbendazole.
Embodiment 65: The method according to embodiment 64, wherein the active kibble includes:
between about 30 mg of mivorilaner/kg of active kibble to about 350 mg of mivorilaner/kg of active kibble;
between about 0.05 mg of moxidectin/kg of active kibble to about 1.0 mg of moxidectin/kg of active kibble; and
between about 30 mg of fenbendazole/kg of active kibble to about 500 mg of fenbendazole/kg of active kibble.
Embodiment 66: The method according to embodiment 64, wherein the active kibble includes:
between about 60 mg of mivorilaner/kg of active kibble to about 200 mg of mivorilaner/kg of active kibble;
between about 0.08 mg of moxidectin/kg of active kibble to about 0.6 mg of moxidectin/kg of active kibble; and
between about 60 mg of fenbendazole/kg of active kibble to about 300 mg of fenbendazole/kg of active kibble.
Embodiment 67: A formulation, including:
an admixture maintained at a temperature between about 40.0° C. and about 65.0° C., the admixture including:
an edible carrier;
an optional surface-active agent; and
an active ingredient.
Embodiment 68: The formulation according to embodiment 67, wherein the admixture is in one or more of the following physical states: a solution, a suspension, an emulsion, a reverse emulsion/vesicle, a solid.
Embodiment 69: The formulation of embodiments 67-68, wherein at least one of the surface-active agents is lecithin.
Embodiment 70: The formulation of embodiments 67-69, wherein at least one of the surface-active agents is tween 80.
Embodiment 71: The formulation of embodiments 67-70, wherein at least one of the surface-active agents is a polysorbate.
Embodiment 72: The formulation of embodiments 67-71, wherein the edible fat is a fish oil.
Embodiment 73: The formulation of embodiments 67-71, wherein the edible fat is a chicken fat.
Embodiment 74: The formulation of embodiments 67-71, wherein the edible fat is a turkey fat.
Embodiment 75: The formulation according to embodiments 67-75, wherein the admixture includes between about 0.5 wt. % to about 10 wt. % of lecithin.
Embodiment 76: The formulation according to embodiments 67-75, wherein the admixture includes between about 1.0 wt. % to about 6.0 wt. % of lecithin.
Embodiment 77: The formulation according to embodiments 67-75, wherein the admixture includes between about 2.0 wt. % to about 5.0 wt. % of lecithin.
Embodiment 78: The formulation according to embodiments 67-75, wherein the admixture includes between about 3.5 wt. % to about 4.5 wt. % of lecithin.
Embodiment 79: The formulation according to embodiments 67-78, wherein the admixture includes between about 90.0 wt. % to about 99.5 wt. % edible fat.
Embodiment 80: The formulation according to embodiments 67-78, wherein the admixture includes between about 94.0 wt. % to about 99.0 wt. % edible fat.
Embodiment 81: The formulation according to embodiments 67-78, wherein the admixture includes between about 95.0 wt. % to about 98.0 wt. % edible fat.
Embodiment 82: The formulation according to embodiments 67-78, wherein the admixture includes between about 95.5 wt. % to about 96.5 wt. % edible fat.
Embodiment 83: The formulation according to embodiments 67-78, wherein the edible fat is chicken fat.
Embodiment 84: The formulation according to embodiments 67-83, wherein the active ingredient is mivorilaner.
Embodiment 85: The formulation according to embodiment 84, wherein the admixture includes between about 0.5 mg of mivorilaner/g of admixture to about 16.0 mg of mivorilaner/g of admixture.
Embodiment 86: The formulation according to embodiment 84, wherein the admixture includes between about 0.8 mg of mivorilaner/g of admixture to about 8.0 mg of mivorilaner/g of admixture.
Embodiment 87: The formulation according to embodiment 84, wherein the admixture includes between about 1.5 mg of mivorilaner/g of admixture to about 4.0 mg of mivorilaner/g of admixture.
Embodiment 88: The formulation according to embodiments 67-83, wherein the active ingredient is moxidectin.
Embodiment 89: The formulation according to embodiment 67-83, wherein the admixture includes between about 0.0005 mg of moxidectin/g of admixture to about 16.0 mg of moxidectin/g of admixture.
Embodiment 90: The formulation according to embodiment 67-83, wherein the admixture includes between about 0.001 mg of moxidectin/g of admixture to about 2.0 mg of moxidectin/g of admixture.
Embodiment 91: The formulation according to embodiment 67-83, wherein the admixture includes between about 0.0015 mg of moxidectin/g of admixture to about 0.1 mg of moxidectin/g of admixture.
Embodiment 92: The formulation according to embodiment 67-83, wherein the admixture includes about 0.002 mg of moxidectin/g of admixture.
Embodiment 93: The formulation according to embodiments 84-87, wherein the active ingredient further includes moxidectin.
Embodiment 94: The formulation according to embodiment 93, wherein the admixture includes between about 0.0005 mg of moxidectin/g of admixture to about 16.0 mg of moxidectin/g of admixture.
Embodiment 95: The formulation according to embodiment 93, wherein the admixture includes between about 0.001 mg of moxidectin/g of admixture to about 2.0 mg of moxidectin/g of admixture.
Embodiment 96: The formulation according to embodiment 93, wherein the admixture includes between about 0.0015 mg of moxidectin/g of admixture to about 0.1 mg of moxidectin/g of admixture.
Embodiment 97: The formulation according to embodiment 93, wherein the admixture includes about 0.002 mg of moxidectin/g of admixture.
Embodiment 98: The formulation according to embodiments 67-83, wherein the active ingredient is cyclosporin.
Embodiment 99: The formulation according to embodiment 98, wherein the admixture includes between about 2.0 mg of cyclosporin/g of admixture to about 8.0 mg of cyclosporin/g of admixture.
Embodiment 100: The formulation according to embodiment 98, wherein the admixture includes between about 4.0 mg of cyclosporin/g of admixture to about 8.0 mg of cyclosporin/g of admixture.
Embodiment 101: The formulation according to embodiment 98, wherein the admixture includes between about 5.0 mg of cyclosporin/g of admixture to about 7.9 mg of cyclosporin/g of admixture.
Embodiment 102: The formulation according to embodiment 98, wherein the admixture includes about 7.9 mg of cyclosporin/g of admixture.
Embodiment 103: The formulation according to embodiment 67-83, wherein the active ingredient is fenbendazole.
Embodiment 104: The formulation according to embodiment 103, wherein the admixture includes between about 0.5 mg of fenbendazole/g of admixture to about 3.0 mg of fenbendazole/g of admixture.
Embodiment 105: The formulation according to embodiment 103, wherein the formulation includes between about 0.75 mg of fenbendazole/g of admixture to about 2.0 mg of fenbendazole/g of admixture.
Embodiment 106: The formulation according to embodiment 103, wherein the formulation includes about 1.6 mg of fenbendazole/g of admixture.
Embodiment 107: The formulation according to embodiments 67-83, wherein the active ingredient is gabapentin.
Embodiment 108: The formulation according to embodiment 107, wherein the admixture includes between about 5.0 mg of gabapentin/g of admixture to about 30.0 mg of gabapentin/g of admixture.
Embodiment 109: The formulation according to embodiment 107, wherein the admixture includes between about 7.5 mg of gabapentin/g of admixture to about 20.0 mg of gabapentin/g of admixture.
Embodiment 110: The formulation according to embodiment 107, wherein the admixture includes about 15.9 mg of gabapentin/g of admixture.
Embodiment 111: A dry carrier admixture, including:
a dry edible carrier;
an optional solid dispersing agent; and
an active ingredient, wherein the dry carrier is mixed with the active ingredient and optionally with the surface-active agent.
Embodiment 112: The admixture, according to embodiment 111, wherein the dry carrier is selected from the group consisting of: pea flour, soy flour, rice flour and wheat flour.
Embodiment 113: The admixture according to embodiment 111, wherein the dry carrier is pea flour.
Embodiment 114: The admixture according to embodiment 111, wherein the dry carrier is soy flour.
Embodiment 115: The admixture according to embodiment 111, wherein the dry carrier is wheat flour.
Embodiment 116: The admixture according to embodiments 111-115, wherein the optional solid dispersing agent is selected from the group consisting of: polyvinyl pyrrolidone, sodium hexametaphosphate, lignosulfonates, and Na-EDTA.
Embodiment 117: The admixture according to embodiments 111-115, wherein the optional solid dispersing agent is polyvinyl pyrrolidone.
Embodiment 118: The admixture according to embodiments 111-115, wherein the optional solid dispersing agent is sodium hexametaphosphate.
Embodiment 119: The admixture according to embodiments 111-115, wherein the optional solid dispersing agent is lignosulfonates.
Embodiment 120: The admixture according to embodiments 111-115, wherein the optional solid dispersing agent is Na-EDTA.
Embodiment 121: The dry carrier admixture, according to embodiments 111-120, wherein the active ingredient is fenbendazole.
Embodiment 122: The dry carrier admixture, according to embodiment 121, wherein the admixture includes between about 1.0 mg of fenbendazole/g of dry carrier to about 100 mg of fenbendazole/g of dry carrier.
Embodiment 123: The dry carrier admixture, according to embodiment 121, wherein the admixture includes between about 1.0 mg of fenbendazole/g of dry carrier to about 50 mg of fenbendazole/g of dry carrier.
Embodiment 124: The dry carrier admixture, according to embodiment 121, wherein the admixture includes between about 10.0 mg of fenbendazole/g of dry carrier to about 14.0 mg of fenbendazole/g of dry carrier.
Embodiment 125: The dry carrier admixture, according to embodiments 111-120, wherein the active ingredient is metformin.
Embodiment 126: The dry carrier admixture, according to embodiment 125, wherein the admixture includes between about 50.0 mg of metformin/g of dry carrier to about 1000.0 mg of metformin/g of dry carrier.
Embodiment 127: The dry carrier admixture, according to embodiment 125, wherein the admixture includes between about 100.0 mg of metformin/g of dry carrier to about 500.0 mg of metformin/g of dry carrier.
Embodiment 128: The dry carrier admixture, according to embodiment 125, wherein the admixture includes between about 200.0 mg of metformin/g of dry carrier to about 300.0 mg of metformin/g of dry carrier.
Embodiment 129: A method of making an animal feed, including:
a) providing at least one active ingredient, in the form of a liquid, or a solid,
b) supplying a dry or semi-dry kibble; and
c) adding the active ingredient to the kibble to form an active kibble.
Embodiment 130: The method according to embodiment 129, further including:
d) applying a coating to the kibble before adding the active ingredient to the kibble to form the active kibble.
Embodiment 131: The method according to embodiments 129-130, further including:
e) tumbling a batch of the active kibble to create a substantially homogenous batch of the active kibble.
Embodiment 132: The method according to embodiment 131, further including repeating one or more of steps d) and e).
Embodiment 133: The methods according to embodiments 129-132, further including exposing the batch of active kibble to a vacuum.
Embodiment 134: The methods according to embodiments 129-133, wherein the active ingredient is substantially a solid when it is added to the kibble.
Embodiment 135: The method according to embodiment 134, where in the substantially dry solid active ingredient is mixed with an anti-caking agent before it is applied to the kibble.
Embodiment 136: The method according to embodiment 135, wherein the anti-caking agent is at least one agent selected from the group consisting of: aluminum calcium silicate, attapulgite clay, calcium silicate, calcium stearate, diatomaceous earth, magnesium silicate, sodium aluminosilicate, hydrated sodium calcium aluminosilicate, tricalcium silicate, hydrophobic silica, iron ammonium citrate, kaolin, montmorillonite clays, sodium silico aluminate and yellow prussiate of soda.
Embodiment 137: The method according to embodiments 130-136, wherein the coating is a synthetic or naturally occurring edible fat.
Embodiment 138: The method according to embodiment 137, wherein the edible fat is a vegetable fat.
Embodiment 139: The method according to embodiment 137, wherein the edible fat is an animal fat selected from the group consisting of; bovine fat, avian fat, ovine fat, porcine fat, and fish oil.
Embodiment 140: The method according to embodiments 129-139, wherein the coating includes a surface-active agent.
Embodiment 141: The method according to embodiment 140, wherein the surface-active agents is amphiphilic.
Embodiment 142: The method according to embodiment 140, wherein the surface-active agent is a surfactant.
Embodiment 143: The method according to embodiments 140, wherein the surface-active agent is a dispersant.
Embodiment 144: The method according to embodiments 129-143, wherein the coating is one or more of the following: a solution; a reverse emulsion/vesicle; or a suspension.
Embodiment 145: The method according to embodiments 129-144, wherein the active kibble further includes at least one palatant.
Embodiment 146: The method according to embodiments 129-145, wherein the active kibble further includes at least one edible coloring agent.
Embodiment 147: The method according to embodiments 129-146, wherein at least a portion of the surface of the kibble is hydrophobic.
Embodiment 148: The method according to embodiments 129-147, wherein at least a portion of the surface of the kibble is hydrophilic.
Embodiment 149: The method according to embodiments 129-148, wherein at least a portion of the surface of the kibble is porous.
Embodiment 150: The method according to embodiments 129-149, further including pre-treating the kibble to increase the kibble's affinity for the active ingredient.
Embodiment 151: The method according to embodiments 129-150, wherein the active ingredient is one or more compounds selected from the group consisting of: an anthelmintic, an acaricide, a fungicide; an antiparasitic compound, an insecticide, an antibiotic, a miticide, an analgesic, and a metabolic regulator.
Embodiment 152: The method according to embodiments 129-150, wherein the active ingredient is mivorilaner.
Embodiment 153: The method according to embodiments 129-152, further including heating the coating greater than 40.00 C before it is mixed with the surface active agent.
Embodiment 154: The method according to embodiments 130-153, wherein the coating is maintained between about 40.00 C to about 65.00 C until it is applied to the feed.
Embodiment 155: The method according to embodiments 130-154, wherein the coating includes between about 0.5 wt. % to about 10 wt. % of a surface active agent.
Embodiment 156: The method according to embodiments 130-154, wherein the coating includes between about 1.0 wt. % to about 6.0 wt. % of a surface active agent.
Embodiment 157: The method according to embodiments 130-154, wherein the coating includes between about 2.0 wt. % to about 5.0 wt. % of a surface active agent.
Embodiment 158: The method according to embodiments 130-154, wherein the coating includes between about 3.5 wt. % to about 4.5 wt. % of a surface active agent.
Embodiment 159: The method according to embodiments 130-158, wherein the coating includes between about 90.0 wt. % to about 99.5 wt. % of an edible fat.
Embodiment 160: The method according to embodiments 130-158, wherein the coating includes between about 94.0 wt. % to about 99.0 wt. % of an edible fat.
Embodiment 161: The method according to embodiments 130-158, wherein the coating includes between about 95.0 wt. % to about 98.0 wt. of an edible fat.
Embodiment 162: The method according to embodiment 130-158, wherein the coating includes between about 95.5 wt. % to about 96.5 wt. % of an edible fat.
Embodiment 163: The method according to embodiments 159-162, wherein the edible fat is chicken fat.
Embodiment 164: The method according to embodiments 129-163, wherein the active ingredient is mivorilaner.
Embodiment 165: The method according to embodiments 129-164, wherein the active kibble includes between about 30 mg of mivorilaner/kg of active kibble to about 350 mg of mivorilaner/kg of active kibble.
Embodiment 166: The method according to embodiments 129-164, wherein the active kibble includes between about 50 mg of mivorilaner/kg of active kibble to about 250 mg of mivorilaner/kg of active kibble.
Embodiment 167: The method according to embodiments 129-164, wherein the active kibble includes between about 60 mg of mivorilaner/kg of active kibble to about 200 mg of mivorilaner/kg of active kibble.

### BRIEF DESCRIPTION OF THE FIGURES

The above-mentioned aspects of exemplary embodiments will become more apparent and will be better understood by reference to the following description of the embodiments taken in conjunction with the accompanying drawings, wherein:

FIG. 1 shows a schematic diagram of a system for applying to a dry feed a coating that includes an active ingredient in a solution.

FIG. 2 shows a schematic diagram of a system for applying to a dry feed a coating that includes an active ingredient in a suspension.

FIG. 3 shows a schematic diagram of a system for applying to a dry feed a coating that includes an active ingredient in solid form.

FIG. 4 shows a schematic diagram of a system for applying to a dry feed a coating that includes an active ingredient in a reverse emulsion.

FIG. 5 shows a graph of the (%) transmissitivity as a function of temperature determined for an admixture of 8 mg of mivorilaner/g of either chicken fat (CF) or chicken fat that includes 4% lecithin, determination made at a ramp rate of 0.1° C./min.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

As used in the following, the terms “have,” “comprise” or “include” or any arbitrary grammatical variations thereof are generally open-ended terms. Thus, these terms may both refer to a situation in which, besides the feature introduced by these terms, no further features happen to be present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once, if at all, when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “surface active agent,” “edible carrier,” and “active ingredient,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

Unless explicitly stated otherwise, or clearly implied otherwise, the term ‘about’ means plus or minus 10 percent of the stated value, for example, about 1.0 includes 0.9 to 1.1.

All ratios, percentages, and parts discussed herein are “by weight” unless otherwise specified.

### Definitions

The term “kibble” as used herein is a broad term and is to be given its ordinary and customary meaning to a person of ordinary skill in the art and is not to be limited to a special or customized meaning. The term “kibble” specifically may refer, without limitation, to dry or semi-dry pellets of pet food. Kibble may be enrobed or unenrobed. The pellets may be of any shape or size that is amenable for their use in a particular feed. Kibble has a surface and an interior, the kibble, especially the surface of the kibble may be porous. Portions of the kibble pellet, including for example the surface may be hydrophobic or hydrophilic. The term kibble is in no way limited by its composition. For example, kibble may be comprised of any of the following: meat, meat by-products, vegetable matter such as processed or unprocessed grains, vitamins, minerals, compounds that make the kibble more or less palatable, preservatives, anti-bacterial and/or anti-fungal agents, antioxidants, and the like.

Types of kibbles useful for the practice of the invention include kibble formed by extruding a feed through an extruder. The kibble may include a mixture of nutritional elements, notably lipids, proteins and carbohydrates, in quality and in quantity suitable for the oral feeding of a particular species and/or variety of animal, including all life stages, lifestyles and life conditions. Typical components of the feed are known in the art and include one or more of the following: cereals such as rice, wheat, barley, oats, corn, and/or soy; vegetables such as peas, carrots, sugar beets; vegetable oils such as sunflower, safflower, cottonseed, soybean, and rape seed; eggs or portions thereof; proteins and/or fats from animals such as birds, fish, and mammals. The kibble is an oral veterinary composition and preferably includes the nutrients deemed necessary for the health and well-being of the target animal.

A kibble may be coated or uncoated. Coatings applied to kibble include coatings that change the organoleptic properties of the kibble, the color of the kibble, the stability of the kibble, the texture of the kibble, and as discussed herein compounds that include various ingredients that change the nutritional content of the kibble, the caloric value of the kibble, various pharmacologically active ingredients, some of which are disclosed herein. Coatings for kibble may include carriers that have a nutritional value, such as plant or animal fats. Coatings may also include compounds that alter the physio-chemical properties of the coating, such as wetting agents, surfactants, emulsifiers, and the like.

The term “carrier” as used herein is a broad term and is to be given its ordinary and customary meaning to a person of ordinary skill in the art and is not to be limited to a special or customized meaning. The term “carrier” specifically may refer, without limitation, to triacetin, animal fat, beef tallow, lard, vegetable oil, fatty acid esters of edible fats, mono or diglycerides of fatty acid esters, poultry fat, chicken fat, duck fat, silicon oil, fish oil or some combination thereof. One type of carrier is a ‘dry carrier’ which may be in the form particles and may be found in or comprise flours.

Carriers that may be included in kibble coatings include, but are not limited to, fats derived from mammals, fish, birds, and plants. Some coatings may include comestible fats formed from petroleum or hydrocarbons.

The term “surface active agents” as used herein is a broad term and is to be given its ordinary and customary meaning to a person of ordinary skill in the art and is not to be limited to a special or customized meaning. The term “surface active agents” specifically may refer, without limitation, to surfactants, dispersing agents, detergent, and the like or combinations thereof, including, for example, lecithin, polysorbates, glycerol monooleate or other fatty acids/esters, sorbitan fatty acid esters such as Tweens, lignosulfonates, nonionic block copolymers or combinations thereof.

One surface-active agent that can be used in some embodiments of this disclosure is lecithin. Sources of lecithin include, but are not limited to, soybeans, sunflowers, eggs, wheat germ, peanuts, liver, rapeseed, corn and the like.

The term “solution” as used herein is a broad term and is to be given its ordinary and customary meaning to a person of ordinary skill in the art and is not to be limited to a special or customized meaning. The term “solution” specifically may refer, without limitation, to a homogenous mixture of one or more solutes dissolved in a solvent. As used herein some fluids are solvents.

The term “suspension” as used herein is a broad term and is to be given its ordinary and customary meaning to a person of ordinary skill in the art and is not to be limited to a special or customized meaning. The term “suspension” specifically may refer, without limitation, to a heterogeneous mixture of a fluid that contains solid particles. Usually, the solid particles are large enough to be capable of forming a sediment enriched in the solid particles.

The term “emulsion” as used herein is a broad term and is to be given its ordinary and customary meaning to a person of ordinary skill in the art and is not to be limited to a special or customized meaning. The term “emulsion” specifically may refer, without limitation, to a mixture of two or more immiscible liquids, wherein one of the liquids contains a dispersion of one of more of the other liquids. In most emulsions between a water and a hydrophobic liquid, the continuous phase is the hydrophilic/aqueous phase.

The term “reverse emulsion/vesicle” as used herein is a broad term and is to be given its ordinary and customary meaning to a person of ordinary skill in the art and is not to be limited to a special or customized meaning. The term “reverse emulsion/vesicle” specifically may refer, without limitation, to a mixture of two or more immiscible liquids, wherein one of the liquids contains a dispersion of one of more of the other liquids. In most reverse emulsion/vesicle between a water and a hydrophobic liquid, the continuous phase is the hydrophobic/non-aqueous phase.

Types of “surfactants” that can be used to practice some embodiments of this disclosure include cationic surfactants, anionic surfactants, nonionic surfactants, and amphoteric surfactants.

The term “non-ionic surfactants” as used herein is a broad term and is to be given its ordinary and customary meaning to a person of ordinary skill in the art and is not to be limited to a special or customized meaning. The term “non-ionic surfactants” specifically may refer, without limitation, to polyethylene glycols, poly (alkylene-oxide) block copolymers (e.g., PEO-PPO-PEO, PPO-PEO-PPO, PBO-PEO, and Tetronic); oligomeric alkyl-ethylene oxides (e.g., Brij and Tergitol); alkyl-phenol pol-ethylenes (e.g., Tween); and sorbitan esters (e.g., Span).

The term “cationic surfactants” as used herein is a broad term and is to be given its ordinary and customary meaning to a person of ordinary skill in the art and is not to be limited to a special or customized meaning. The term “cationic surfactants” specifically may refer, without limitation, to triethylamine hydrochloride; octenidine dihydrochloride; cetromonium bromide; cetypyridium chloride; adogen; benzethonium chloride; and dimethyldioctadecylammonium chloride.

The term “anionic surfactants” as used herein is a broad term and is to be given its ordinary and customary meaning to a person of ordinary skill in the art and is not to be limited to a special or customized meaning. The term “anionic surfactants” specifically may refer, without limitation, to carboxylic acids; sodium stearate, sodium lauroyl sarcosinate; cholic acid; deoxycholic acid; glycolic acid ethoxylate 4-tert-butylpheny ether; glycolic acid ethoxylate laurylphenyl ether; zonyl fluorosurfactant; and glycolic acid ethoxylate oleyl ether.

The term “amphoteric surfactants” as used herein is a broad term and is to be given its ordinary and customary meaning to a person of ordinary skill in the art and is not to be limited to a special or customized meaning. The term “amphoteric surfactants” specifically may refer, without limitation, to lauryl betaine; lauroyl sarcosinate; lauryl sultaine; laulryamidopropylbetaine; and lauryldimehtylamide oxide.

The terms “solid dispersing agents” and “dispersing agents” are used interchangeably. As used herein, these are broad terms and are to be given their ordinary and customary meaning to a person of ordinary skill in the art and are not to be limited to a special or customized meaning. Dispersion may be achieved by mechanical, steric or electronic means, or a combination thereof. The terms “solid dispersing agents” and “dispersing agents” specifically may refer, without limitation, to polyvinyl pyrrolidone, sodium hexametaphosphate, lignosulfonates, and Na-EDTA.

The terms “active material” and “active ingredient” are used interchangeably herein and as used herein are broad terms to be given their ordinary and customary meaning to a person of ordinary skill in the art and are not to be limited to a special or customized meaning. The terms “active material” and “active ingredient” specifically may refer, without limitation, to any ingredient or combination of ingredients included in a feed primarily for to prevent or control infestations of parasites, e.g., fleas, ticks, helminths, lice, mites, etc. For example, an active ingredient may be a therapeutic agent used to diagnose, cure, treat, or prevent a condition. A therapeutic purpose or effect may include any effect or action of the active ingredient intended to diagnose, cure, mitigate, treat or prevent disease or affect the structure or any function of the body.

The term “controlling a tick infestation” as used herein is a broad term and is to be given its ordinary and customary meaning to a person of ordinary skill in the art and is not to be limited to a special or customized meaning. The term “controlling a tick infestation” specifically may refer, without limitation, to preventing, treating, repelling, minimizing or eliminating an infestation by ticks on a mammal.

The term “tick” refers to any member of the order Ixodida. The term “tick” includes the egg, larval, nymph, and adult stages of development. More particularly, the term “tick” includes ticks of the families Ixodidae and Argasidac. More particularly, the term “tick” includes species of the genera Africaniella, Amblyomma, Anomalohimalaya, Bothriocroton, Dermacentor, Haemaphysalis, Hyalomma, Ixodes, Margaropus, Nosomma, Rhipicentor, Rhipicephalus, Antricola, Argas, Nothoaspis, Ornithodoros, and Otobius.

The terms “controlling a parasitic worm infestation” and “controlling a helminth infestation” as used herein are broad terms and to be given their ordinary and customary meaning to a person of ordinary skill in the art and are not to be limited to a special or customized meaning. The terms “controlling a parasitic worm infestation” and “controlling a helminth infestation” specifically may refer, without limitation, to preventing, treating, minimizing or eliminating an infestation by parasitic worms or helminths in a mammal.

The terms “parasitic worms” and “helminths” are used interchangeably herein and are broad terms and to be given their ordinary and customary meaning to a person of ordinary skill in the art and are not to be limited to a special or customized meaning. The terms “parasitic worms” and “helminths” specifically may refer, without limitation, to members of the phyla Annelida, Platyhelminthes, Nematoda and Acanthocephala. The terms “parasitic worms” and “helminths” include the egg, larval and adult stages of development.

The term “mammal” as used herein is a broad term and is to be given its ordinary and customary meaning to a person of ordinary skill in the art and is not to be limited to a special or customized meaning. The term “mammal” specifically may refer, without limitation, to any member of the class Mammalia. In particular, it may refer to wild mammals, such as wolves, coyotes, jackals, deer, elk, moose, reindeer, and the like. It may also refer to farm animals, such as cows, sheep, pigs, bison, horses, and the like. It may also refer to companion animals.

The term “companion animal” as used herein is a broad term and is to be given its ordinary and customary meaning to a person of ordinary skill in the art and is not to be limited to a special or customized meaning. The term “companion animal” specifically may refer, without limitation, to any domestic animal that suitable for keeping as a pet. This includes, but is not limited to, equines, canines and felines. Still other companion animals include hamsters, gerbils, mice, guinea pigs, ferrets, rabbits, etc.

The term “canine” as used herein is a broad term and is to be given its ordinary and customary meaning to a person of ordinary skill in the art and is not to be limited to a special or customized meaning. The term “canine” specifically may refer, without limitation, to any member of the genus Canis, which includes such species as wolves, dogs, coyotes and jackals.

The term “feline” as used herein is a broad term and is to be given its ordinary and customary meaning to a person of ordinary skill in the art and is not to be limited to a special or customized meaning. The term “feline” specifically may refer, without limitation, to any member of the subfamily Felidae, which includes such species as the domestic cat, bobcats, wildcats, ocelots, members of the genus Lynx, Pallas's cat and cougars.

The term “therapeutically effective” as used herein is a broad term and is to be given its ordinary and customary meaning to a person of ordinary skill in the art and is not to be limited to a special or customized meaning. The term “therapeutically effective” specifically may refer, without limitation, to a dose or blood level of an active ingredient that is sufficient to control the disease condition or infestation better than if no active ingredient were present. The active ingredient may be present on its own or with one or more additional active substances. Preferably, it controls the disease condition or infestation at around at least 50% better than if no active ingredient were present, and more preferably it controls the disease condition or infestation at about at least 90% better than if no active ingredient were present.

The term “disease state” as used herein is a broad term and is to be given its ordinary and customary meaning to a person of ordinary skill in the art and is not to be limited to a special or customized meaning. The term “disease state” specifically may refer, without limitation, to any disease that affects mammals. For example, “disease state” may include diseases that affect companion animals such as dogs and cats.

Unless explicitly stated otherwise or clearly implied otherwise terms such as “reservoir,” “vessel,” “tank,” “drum,” “container,” “bin,” and the like may be used interchangeably.

The term “first reservoir” as used herein is a broad term and is to be given its ordinary and customary meaning to a person of ordinary skill in the art and is not to be limited to a special or customized meaning. The term “first reservoir” specifically may refer, without limitation, to any appropriately sized and designed container, e.g., tank, plastic lined tote, plastic tote, intermediate bulk container, or drum. A first reservoir may be heated or unheated, agitated or not agitated, rotated or not rotated, for example.

The term “second reservoir” as used herein is a broad term and is to be given its ordinary and customary meaning to a person of ordinary skill in the art and is not to be limited to a special or customized meaning. The term “second reservoir” specifically may refer, without limitation, to any appropriately sized and designed container, e.g., heated tank, plastic lined tote, plastic tote, intermediate bulk container, or drum. A second reservoir may be heated or unheated, agitated or not agitated, rotated or not rotated, for example.

The term “third reservoir” as used herein is a broad term and is to be given its ordinary and customary meaning to a person of ordinary skill in the art and is not to be limited to a special or customized meaning. The term “third reservoir” specifically may refer, without limitation, to any appropriately sized and designed container, e.g., bucket, hopper, pouch, canister, plastic lined fiber, plastic drum, tank, tote, intermediate bulk container or other drum. The third reservoir may be unheated, not agitated, rotated or not rotated.

The term “fourth reservoir” as used herein is a broad term and is to be given its ordinary and customary meaning to a person of ordinary skill in the art and is not to be limited to a special or customized meaning. The term “fourth reservoir” specifically may refer, without limitation, to any appropriately sized and designed container, e.g., bucket, hopper, pouch, canister, plastic lined fiber, plastic drum, tank, tote, intermediate bulk container or other drum.

The term “fifth reservoir” as used herein is a broad term and is to be given its ordinary and customary meaning to a person of ordinary skill in the art and is not to be limited to a special or customized meaning. The term “fifth reservoir” specifically may refer, without limitation, to any appropriately sized and designed container, e.g., bucket, hopper, pouch, canister, plastic lined fiber, plastic drum, tank, tote, intermediate bulk container or other drum.

The term “kibble reservoir” as used herein is a broad term and is to be given its ordinary and customary meaning to a person of ordinary skill in the art and is not to be limited to a special or customized meaning. The term “kibble reservoir” specifically may refer, without limitation, to any appropriately sized and designed container, e.g., a surge bin, drum, tank, plastic lined tote, plastic tote, intermediate bulk container, or similar. A kibble reservoir may be heated or unheated, agitated or not agitated, rotated or not rotated, for example.

The term “mixing vessel” as used herein is a broad term and is to be given its ordinary and customary meaning to a person of ordinary skill in the art and is not to be limited to a special or customized meaning. The term “mixing vessel” specifically may refer, without limitation, to a heated or unheated, agitated tank with a mixing and/or shearing blade, a stir paddle, an inline mixer, a high shear mixer, or any appropriately sized mixing apparatus or combinations thereof.

The term “dispersing mechanism” as used herein is a broad term and is to be given its ordinary and customary meaning to a person of ordinary skill in the art and is not to be limited to a special or customized meaning. The term “dispersing mechanism” specifically may refer, without limitation, to a spray bar, a nozzle or nozzles, a duck bill, a fixed or rotating dispenser, or some combination thereof.

The term “dispersing tank” as used herein is a broad term and is to be given its ordinary and customary meaning to a person of ordinary skill in the art and is not to be limited to a special or customized meaning. The term “dispersing tank” specifically may refer, without limitation, to a homogenizer, a high shear mixer, an in-line mixer, or any such mechanical instrument.

The term “blending mechanism” as used herein is a broad term and is to be given its ordinary and customary meaning to a person of ordinary skill in the art and is not to be limited to a special or customized meaning. The term “blending mechanism” specifically may refer, without limitation, to a ribbon blender, paddle blender, enrober, coater, rotating drum coater, tumble drum, screw conveyer coater, vacuum coater, atmospheric coater, mixer, or some combination thereof.

The term “conveyor” as used herein is a broad term and is to be given its ordinary and customary meaning to a person of ordinary skill in the art and is not to be limited to a special or customized meaning. The term “conveyor” specifically may refer, without limitation, to a screw conveyer, elevator, vibratory conveyer with or without a screen, a conveyor belt or any combination thereof.

The term “dry additive feeder” as used herein is a broad term and is to be given its ordinary and customary meaning to a person of ordinary skill in the art and is not to be limited to a special or customized meaning. The term “dry additive feeder” specifically may refer, without limitation, to a mechanism used to distribute a solid. The distribution mechanism may be in the form of a single point, curtain, atomizer, a sifter or some combination thereof.

The term “tumbling” as used herein is a broad term and is to be given its ordinary and customary meaning to a person of ordinary skill in the art and is not to be limited to a special or customized meaning. The term “tumbling” specifically may refer, without limitation, to any process for homogenizing a coating and/or active ingredient on and/or in a kibble.

The term “anti-caking agent” as used herein is a broad term and is to be given its ordinary and customary meaning to a person of ordinary skill in the art and is not to be limited to a special or customized meaning. The term “anti-caking agent” specifically may refer, without limitation, to, e.g., aluminum calcium silicate, attapulgite clay, calcium silicate, calcium stearate, diatomaceous earth, magnesium silicate, sodium aluminosilicate, hydrated sodium calcium aluminosilicate, tricalcium silicate, hydrophobic silica, iron ammonium citrate, kaolin, montmorillonite clays, sodium silico aluminate, yellow prussiate of soda, etc.

The terms “percent inclusion” or “inclusion rate” as used herein is a broad term and is to be given its ordinary and customary meaning to a person of ordinary skill in the art and is not to be limited to a special or customized meaning. The terms “percent inclusion” or “inclusion rate” specifically may refer, without limitation, to, e.g., the amount of active ingredient premixes or admixtures (solutions, suspensions, dry premixes, or combinations thereof) that are applied to a given amount of feed to result in a final medicated feed with the desired amount of active pharmaceutical ingredient(s).

### Kibble

Some embodiments of this disclosure comprise a “daily feed.” The feed may be in the form of an animal feed, kibble, snack, treat or other supplemental feed that may be administered daily or substantially daily.

Kibble that can be used to practice some embodiments of this disclosure are mixtures of nutritional elements, e.g., lipids, proteins and carbohydrates, in quality and in quantity suitable for feeding the animals for which the oral composition is intended. In other words, an oral veterinary composition according to this disclosure includes nutrients essential for the animals for which it is intended.

Kibble is generally made by extruding or pressing a nutritional raw material or a mixture of nutritional raw materials. The mixture may include, for example, carbohydrates derived from, e.g., plants or milk and/or of proteins derived from, e.g., plants, animal sources including meat, eggs, fish, and/or a mixture of animal or vegetable fats. The use of kibble is common in the food industry, in particular for making dry or semi-dry feeds intended for animals, in particular for companion animals such as dogs and cats.

The kibble may be treated by various methods including, for example, by vapor deposition, vacuum coating or by spraying the surface with a fat or oil composition that has been liquefied by heating. The temperature of the coating material may be adjusted to alter its physical properties.

The constituents used for preparing a kibble according to this disclosure include, but are not limited to, farinaceous substances, proteinaceous substances, and fats. In certain embodiments, the body of the kibble may comprise mixtures or combinations of at least two of proteins, carbohydrates, fibers, fats, minerals and/or vitamins.

The protein component of kibble may include meat and/or meat byproducts. Meat may include, for example, chicken, pork, lamb or mutton, turkey, beef, goat meat and/or fish. Meat may include skeletal muscle, offal, lungs, kidneys, livers, stomachs, intestines and other organs. Alternatively, or additionally, the protein component of kibble may include milk, eggs, plant-based proteins or synthetically produced proteins.

Commonly used carbohydrates for feed may include substances derived from cereals, especially substances derived from corn, wheat, rice, oats, and/or sorghum.

Substances comprising fibers include fructooligosaccharides, beet pulp, the mannanoligosaccharides, oat fiber, pulp of citrus fruits, carboxymethylcellulose, as well as gums such as gum arabic, guar gum, or apple or tomato pomaces, and mixtures or combinations thereof.

Fats may include fats of animal origin and/or fats of vegetable origin.

Fats of vegetable origin may include vegetable oils such as maize oils, soybean oils, cotton oils, palm oils, coconut oils, sesame oils, sunflower oils, safflower oil, as well as mixtures and combinations thereof.

Mineral substances include sodium selenite, monosodium phosphate, calcium carbonate, potassium chloride, ferrous sulfate, zinc oxide, manganese sulfate, copper sulfate, manganese oxide, potassium iodide, cobalt carbonate bone meal, as well as mixtures and combinations thereof.

Vitamins may include choline chloride, vitamin E, ascorbic acid, vitamin A acetate, calcium pantothenate, pantothenic acid, biotin, thiamine mononitrate, vitamin B12, niacin, riboflavin, inositol, pyridoxine hydrochloride, vitamin D3, folic acid, vitamin C, as well as mixtures and combinations thereof.

The kibble may also include amino acids such as methionine, leucine, lysine, tryptophan, arginine, cysteine, aspartic acid, taurine, as well as mixtures and combinations thereof.

The kibble may include additional substances such as prebiotics, probiotics, functional feed ingredients, antioxidants, stabilizers, binders, thickeners, taste exhausters, flavorings, preservatives, fillers, emulsifiers, sweeteners, buffering agents, dyes, gelling agents and humectants.

### Admixture

In some embodiments of this disclosure, an admixture is formed by adding one or more active ingredients to a pre-formed solution, suspension carrier, reverse emulsion or inactive solid diluent/carrier.

The active ingredient may be preconditioned by treating the active ingredient with a solution of an oily or fatty liquid preconditioned to form waxy granules, by adding water, by adding a wetting agent and allowing the active ingredient to dry, or by incorporation of a solid diluent/carrier.

### Coating Processes for Creating an Active Kibble

An active kibble is a kibble that includes at least one active ingredient, preferably added to the kibble after the kibble has been extruded and dried or substantially dried. Processes as exemplified in the figures and detail below may include adding one or more active ingredients to the kibble. The active ingredients may be added as part of a solution, a suspension, a solid, a reverse emulsion/vesicle, or some combination thereof.

One of skill in the art will appreciate that it may be possible to mix active kibble with kibble that is not active to achieve a particular dose.

Process for Applying an Admixture that is a Solution (100)

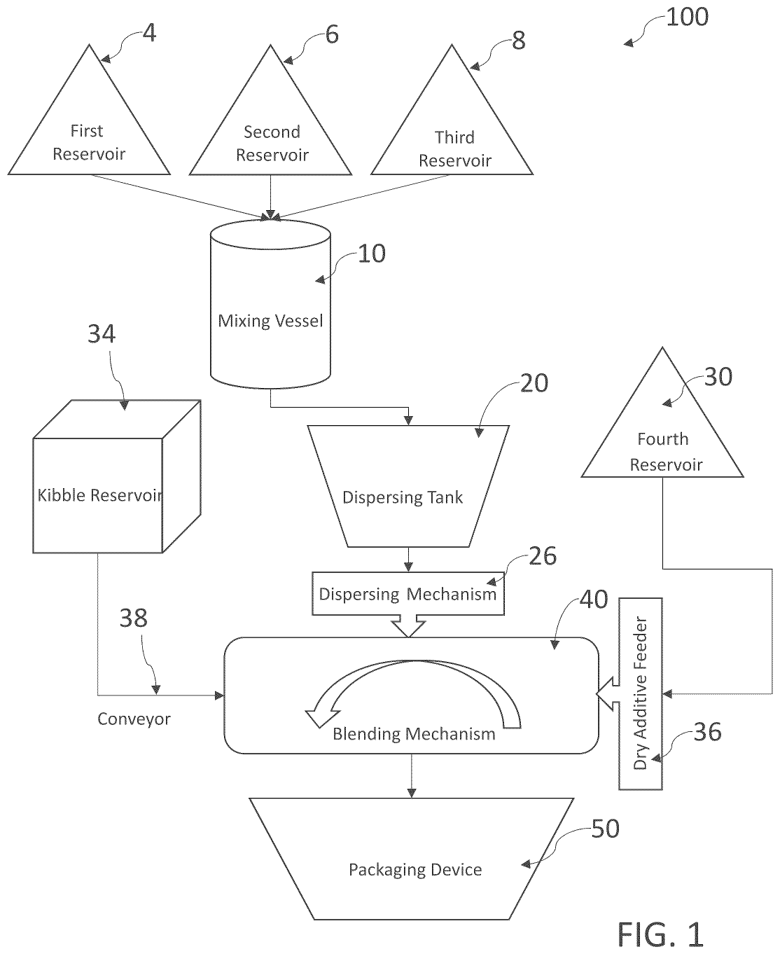

Referring now to FIG. 1. A first reservoir (4) includes a carrier. A second reservoir (6) includes surface-active agent. A third reservoir (8) includes an active ingredient, for example mivorilaner. Any and/or all of reservoirs (4), (6), and (8) may be heated and/or fitted with agitators. Outlets from all three reservoirs connect to mixing vessel (10). Mixing vessel (10) may be optionally equipped with a heater. The outlet from each of reservoirs (4), (6), and (8) may be equipped with a manual or an electrically controlled valve to regulate the amounts of carrier, surface-active agent, and/or active ingredient that flows into mixing tank (10). An outlet from mixing tank (10) may be connected to dispersing tank (20). The outlet from mixing tank (10) connecting to dispersing tank (20) can be equipped with a manual or an electrically controlled valve to regulate the amount of mixture in tank (10) that flows in to dispersing tank (20). Dispersing tank (20) can be optionally heated, agitated, and pressurized. An outlet from dispersing tank (20) connects to dispersing mechanism (26). The flow between dispersing tank (20) and dispersing mechanism (26) may be aided by pressurizing dispersing tank (20), or by pump, or gravity feed as needed.

Still referring to FIG. 1, Dispersing mechanism (26) may connect to a blending mechanism (40). Blending mechanism (40) may be loaded with a kibble suitable for coating with the mixture dispersed by the dispersing mechanism (26). The kibble included in blending mechanism (40) may be stored in kibble reservoir (34) and added either manually or automatically via conveyor (38) to blending mechanism (40). A fourth reservoir (30) that includes at least one palatant connects to a dry additive feeder (36) which connects to blending mechanism (40). Dry additive feeder (36) may dispense a solid either manually or automatically into the bed of blending mechanism (40). The flow of palatant from dry additive feeder (36) to blending mechanism (40) can be equipped with a manual or an electrically controlled valve to regulate the amount of palatant delivered into blending mechanism (40). After the kibble is coated, it can be sent to packaging device (50) for packing into any form acceptable for the delivery of animal feed to a distributor or an end user.

In some embodiments, a second solid active may be added directly to blending mechanism (40) either through dry additive feeder (36) or through a separated dry additive feeder (not shown). Optionally, the coated kibble may pass through a cooling tower (not shown), prior to packaging device (50).

Process for Applying an Admixture that is a Suspension (200)

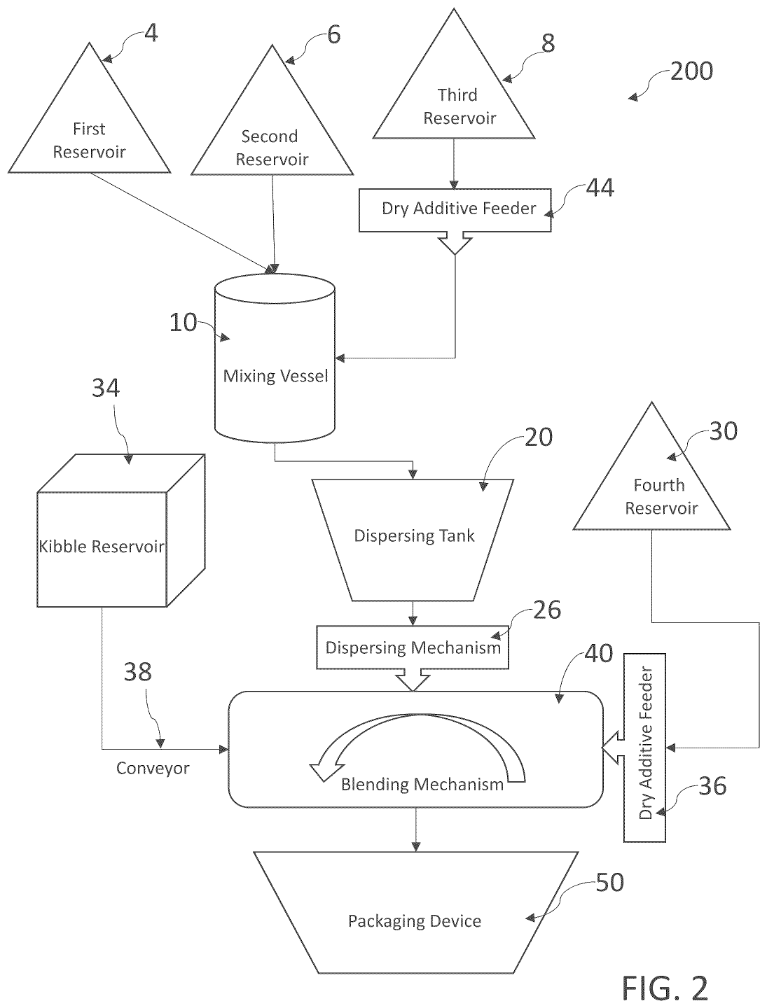

Referring now to FIG. 2, a first reservoir (4) includes a carrier. An optional second reservoir (6) may include a surface-active agent. Any and/or all of reservoirs (4) and (6) may be heated and/or fitted with agitators. Outlets from first and second reservoirs (4, 6) connect to mixing vessel (10). Mixing vessel (10) may be optionally equipped with a heater and/or an agitator. The outlet from first and second reservoirs (4, 6) may be equipped with a manual or an electrically controlled valve to regulate the amounts of carrier and/or surface-active agent that flows into mixing vessel (10). After the carrier and surface-active agent have been sufficiently mixed in mixing vessel (10), the active ingredient in the form of a solid may be added to mixing vessel (10) via dry additive feeder (44) to create a suspension. The dry additive feeder (44) may also be connected to a third reservoir (8) that includes the dry active ingredient. If the active ingredient does not wet or suspend by itself, the active ingredient may be combined with an inactive ingredient, dried, and then placed in third reservoir (8) (components for pre-mixing and drying the active ingredient not shown).

An outlet from mixing vessel (10) may be connected to dispersing tank (20). The outlet from mixing vessel (10) connecting to dispersing tank (20) can be equipped with a manual or an electrically controlled valve to regulate the amount of mixture in vessel (10) that flows in to dispersing tank (20). Dispersing tank (20) can be optionally heated, agitated, and pressurized. Further, dispersing tank (20) may require additional energy input and/or agitation to optimally disperse active ingredient particles. An outlet from dispersing tank (20) connects to dispersing mechanism (26). The flow between dispersing tank (20) and dispersing mechanism (26) may be aided by pressurizing dispersing tank (20), or by pump, or gravity feed as needed.

Still referring to FIG. 2, dispersing mechanism (26) may connect to a blending mechanism (40). Blending mechanism (40) may be loaded with a kibble suitable for coating with the mixture dispersed by the dispersing mechanism (26). The kibble included in blending mechanism (40) may be stored in kibble reservoir (34) and added either manually or automatically via conveyor (38) to blending mechanism (40). A fourth reservoir (30) that includes at least one palatant connects to a dry additive feeder (36) which connects to blending mechanism (40). Dry additive feeder (36) may dispense a solid either manually or automatically into the bed of blending mechanism (40). The flow of palatant from dry additive feeder (36) to blending mechanism (40) can be equipped with a manual or an electrically controlled valve to regulate the amount of palatant delivered into blending mechanism (40). After the kibble is coated, it can be sent to packaging device (50) for packing into any form acceptable for the delivery of animal feed to a distributor or an end user.

In some embodiments, a second solid active may be added directly to blending mechanism (40) either through dry additive feeder (36) or through a separated dry additive feeder (not shown). Optionally, the coated kibble may pass through a cooling tower (not shown), prior to packaging device (50).

Process for Applying an Admixture that is a Solid (300)

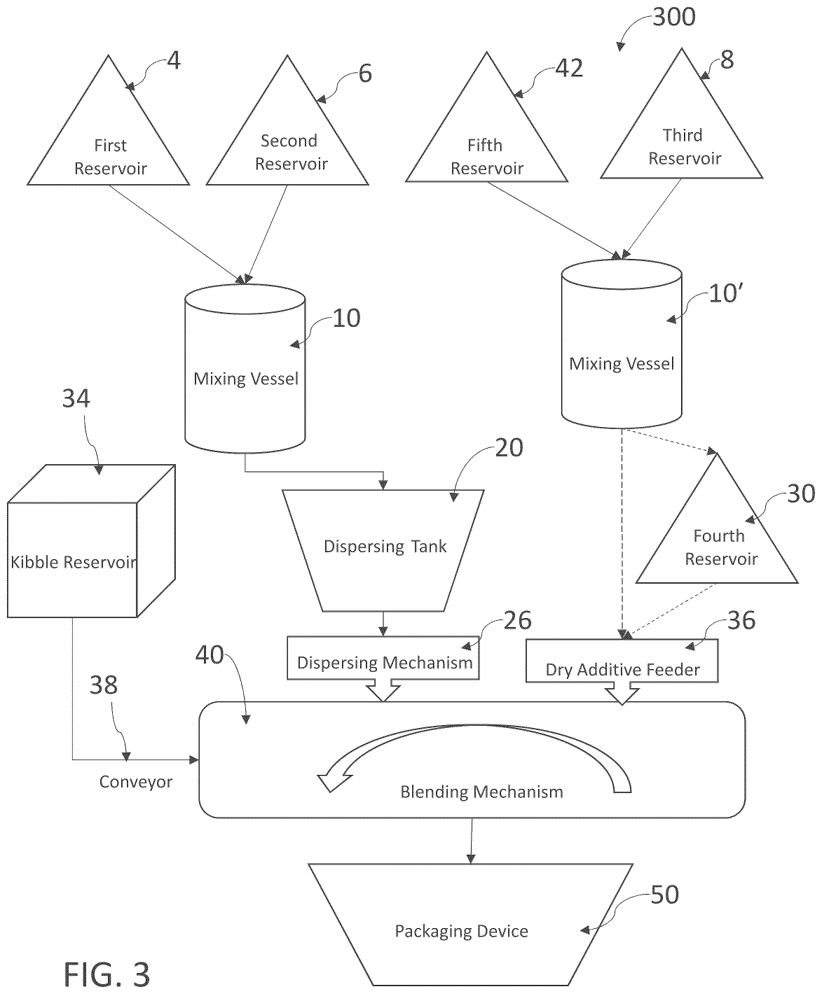

Referring now to FIG. 3. A first reservoir (4) includes a carrier. A second reservoir (6) may include and optional surface-active agent. Any and/or all of reservoirs (4) and (6) may be heated and/or fitted with agitators. Outlets from the first and second reservoirs connect to mixing vessel (10). Mixing vessel (10) may be optionally equipped with a heater. The outlet from reservoirs (4) and (6) may be equipped with a manual or an electrically controlled valve to regulate the amounts of carrier and/or optional surface-active agent that flows into mixing vessel (10). An outlet from mixing vessel (10) may be connected to dispersing tank (20). The outlet from mixing vessel (10) connecting to dispersing tank (20) can be equipped with a manual or an electrically controlled valve to regulate the amount of mixture in vessel (10) that flows in to dispersing tank (20). Dispersing tank (20) can be optionally heated, agitated, and pressurized. An outlet from dispersing tank (20) connects to dispersing mechanism (26).

Mixing a solid active ingredient with a dry carrier may be particularly effective for highly potent solid active ingredients that need to be diluted to provide a lower dosage in the activated kibble. As can be seen in FIG. 3, a third reservoir (8) may contain a solid active ingredient such as, for example, fenbendazole. A fifth reservoir (42) may contain a dry carrier such as, for example, wheat or pea flour. Third reservoir (8) and fifth reservoir (42) may be connected to a mixing vessel (10′). Mixing vessel (10′) may be connected to dry additive feeder (36). Optionally mixing vessel (10′) may be connected to fourth reservoir (30) that includes at least one palatant. It is understood that the palatant and the contents of mixing vessel (10′) may be mixed with one another before applying the mixture of solids to the kibble. Optionally, although not shown, may be a third mixing vessel used to combine the palatant with the solid active ingredient. Fourth reservoir (30) connects to a dry additive feeder (36) which connects to blending mechanism (40). Dry additive feeder (36) may dispense the solid either manually or automatically into the bed of blending mechanism (40). The flow of active ingredient and/or palatant from dry additive feeder (36) to blending mechanism (40) can be equipped with a manual or an electrically controlled valve to regulate the amount of active ingredient and/or palatant delivered into blending mechanism (40).

Still referring to FIG. 3, dispersing mechanism (26) and dry additive feeder (36) may connect to blending mechanism (40). Blending mechanism (40) may be loaded with a kibble suitable for coating with the mixtures dispersed by the dispersing mechanism (26) and dry additive feeder (36). The kibble included in blending mechanism (40) may be stored in kibble reservoir (34) and added either manually or automatically via conveyor (38) to blending mechanism (40). The carrier and optional surface active agent may be dispensed from dispersing mechanism (26) first and the solid active ingredient may be added through dry active feeder (36) after the kibble is initially coated with the carrier and optional surface active agent.

Alternatively, the solid active ingredient may be added through dry active feeder (36) first and the carrier and optional surface active agent may be dispensed from dispersing mechanism (26) after the kibble is initially coated with the solid active ingredient. In another alternative, the carrier and optional surface active agent and the solid active ingredient may be dispensed into blending mechanism (40) simultaneously. In another alternative, the carrier and optional surface active agent may be dispensed from dispersing mechanism (26) first and the solid active ingredient may be added through dry active feeder (36) after the kibble is initially coated with the carrier and surface active agent. The carrier and optional surface active agent may then be dispensed via dispersing mechanism (26) a second time.

After the kibble is coated, it can be sent by vibratory conveyer to packaging device (50) for packing into any form acceptable for the delivery of animal feed to a distributor or an end user. Optionally, the coated kibble may pass through a cooling tower (not shown), prior to packaging device (50).

Process for Applying an Admixture that is a Reverse Emulsion/Vesicle (400)

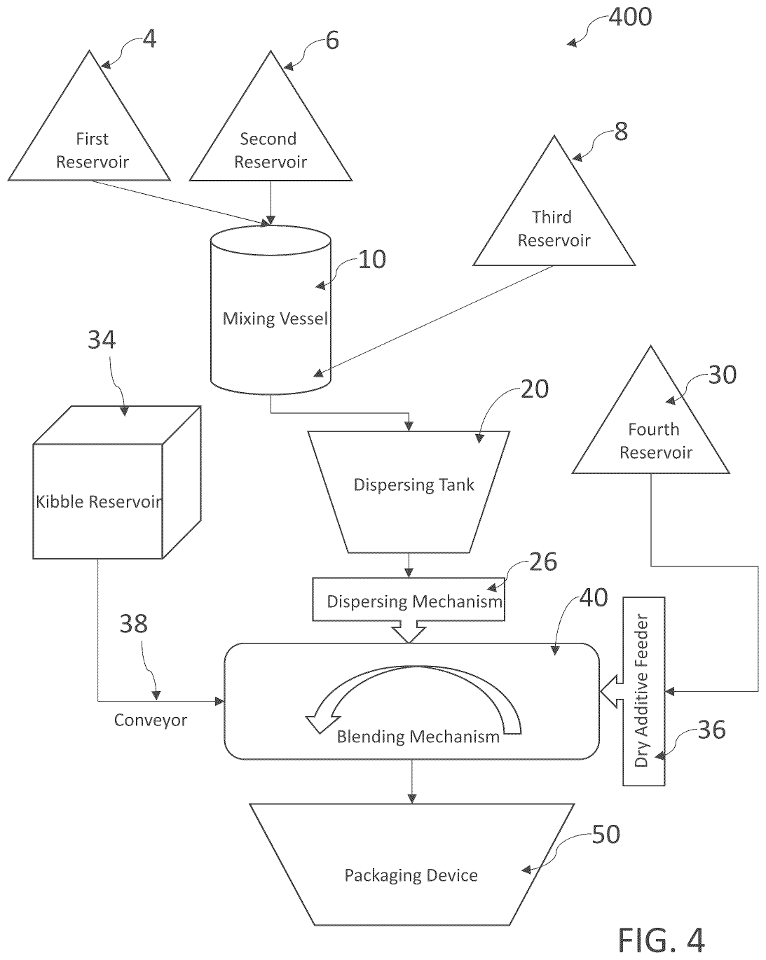

Referring now to FIG. 4, a first reservoir (4) includes a carrier. A second reservoir (6) includes surface-active agent. Any and/or all of reservoirs (4) and (6) may be heated and/or fitted with agitators. Outlets from first and second reservoirs (4, 6) connect to mixing vessel (10). Mixing vessel (10) may be optionally equipped with a heater. The outlet from each first and second reservoir (4, 6) may be equipped with a manual or an electrically controlled valve to regulate the amounts of carrier and/or surface-active agent that flows into mixing vessel (10).

A third reservoir (8) may hold an active ingredient, such as fluoxetine hydrochloride, or diclofenac sodium, that has been mixed with a small amount of water and, optionally, a surface active agent. The water may be less than 5% of the weight of the finished active kibble. More preferably, it may be less than 4% of the weight of the finished active kibble. Still more preferably, the water may be less than 2% of the weight of the finished active kibble. After the carrier and surface-active agent have been sufficiently mixed in mixing vessel (10), the active ingredient in water may be added to mixing vessel (10) from third reservoir (8) to create a reverse emulsion/vesicle.

An outlet from mixing vessel (10) may be connected to dispersing tank (20). The outlet from mixing vessel (10) connecting to dispersing tank (20) can be equipped with a manual or an electrically controlled valve to regulate the amount of mixture in vessel (10) that flows in to dispersing tank (20). Dispersing tank (20) can be optionally heated, agitated, and pressurized. An outlet from dispersing tank (20) connects to dispersing mechanism (26). The flow between dispersing tank (20) and dispersing mechanism (26) may be aided by pressurizing dispersing tank (20), or by pump, or gravity feed as needed.

Still referring to FIG. 4, dispersing mechanism (26) may connect to a blending mechanism (40). Blending mechanism (40) may be loaded with a kibble suitable for coating with the mixture dispersed by the dispersing mechanism (26). The kibble included in blending mechanism (40) may be stored in kibble reservoir (34) and added either manually or automatically via conveyor (38) to blending mechanism (40). A fourth reservoir (30) that includes at least one palatant connects to a dry additive feeder (36) which connects to blending mechanism (40). Dry additive feeder (36) may dispense a solid either manually or automatically into the bed of blending mechanism (40). The flow of palatant from dry additive feeder (36) to blending mechanism (40) can be equipped with a manual or an electrically controlled valve to regulate the amount of palatant delivered into blending mechanism (40). After the kibble is coated, it can be sent to packaging device (50) for packing into any form acceptable for the delivery of animal feed to a distributor or an end user.

In some embodiments, a second solid active may be added directly to blending mechanism (40) either through dry additive feeder (36) or through a separate dry additive feeder (not shown). Optionally, the coated kibble may pass through a cooling tower (not shown), prior to packaging device (50).

### Process for Applying Multiple Admixtures

It is also contemplated that one or more active ingredients may be applied using any of the above-described processes or combinations thereof. For example, an active ingredient may be combined with a surface active agent and applied to the kibble. A carrier, alone or in combination with an active ingredient, may subsequently also be applied to the kibble. It is also possible to add as many layers of one or more admixtures as necessary to achieve an active kibble having a desired amount of a particular active ingredient and/or a desired combination of active ingredients.

### EXAMPLES

### Example 1: Solubility of Active Ingredients in Various Liquid Carriers

To determine the solubility of various active ingredients that could be incorporated into pet food, each active ingredient may be evaluated in a temperature dependent manner using a crystallizer multi-reactor with transmissivity measurements. A non-limiting summary of various experiments with various active ingredients and inactive ingredients is shown below.

Carrier of interest may be prepared as needed and specific active ingredients may be added to the solvents to form mixtures of the solvent and active. Initially, admixtures of carriers and active ingredients for example solvents and actives can be visually inspected to determine if the active disperses or dissolves in the carrier of interest. Indicia that a specific active does not disperse in a given carrier include partitioning of the solid form of the active ingredient to the surface of the carrier, and/or the active ingredient aggregating or agglomerating and either partitioning to the bottom of the carrier or becoming suspended in the test carrier. If an active ingredient does not appear to disperse in a carrier of interest, vigorous mixing and/or heat may be applied to determine if the active ingredient can be made to disperse or dissolve within the test carrier. If mixing and/or heating fails to provide visual evidence that an active ingredient dissolves in the test solvent carrier no further solubility testing of the active ingredient with the test carrier can be conducted.

For active ingredients that appear to at least partially dissolve in a given carrier, the mixtures can be analyzed by transmissivity measurement using a crystallizer multi-reactor. Prior to the transmissivity measurement using a crystallizer multi-reactor with transmissivity capability, carriers may be prepared, as needed, and subsequently tested using a heating cycle over a range of temperatures. The transmissivity data can be recorded over the temperature ranges and used as a blank or reference in subsequent transmissivity measurements.

Typically, initial samples including the active ingredient of interest and the test carrier are not readily soluble at room temperature and may register low percent transmissivity along with visible observations of insolubilized material. When resulting admixtures are heated at a specific heating rate, the percent transmissivity increases as the active ingredient is solubilized. When the solids are completely dissolved, the transmissivity is measured as 100%. When the resulting heated admixture is subsequently cooled, solids may precipitate at a given temperature and cooling rate, which results in a decrease in percent transmissivity. The temperature and time at which precipitation occurs with a specific admixture provides additional information regarding solubility and the combination of parameters needed for selection to prepare feed with the active ingredient of interest.

Experiments can be performed with, for example, mivorilaner, sarolaner, lotilaner, fluralaner, afoxolaner, spinosyn, pyrantel, praziquantel, fenbendazole, diethylcarbamazine, moxidectin, gabapentin, metformin, cyclosporin, and mirtazapine. Carriers include, refined chicken fat, refined chicken fat including lecithin, refined chicken fat including Tween80, refined chicken fat including triacetin, and triacetin may be used as carrier systems over a range of temperatures, ramp rates, stirring rates, and cooling cycles to evaluate stability.

### Mivorilaner Solubility

In an experiment performed for mivorilaner, the initial solubility of mivorilaner was evaluated at concentrations of 2 mg/g, 4 mg/g, 8 mg/g, 16 mg/g, a heating cycle of 0.1° C./minute with 2 heating and cooling cycles over a range of 39° C. to 60° C. while stirring the admixture.

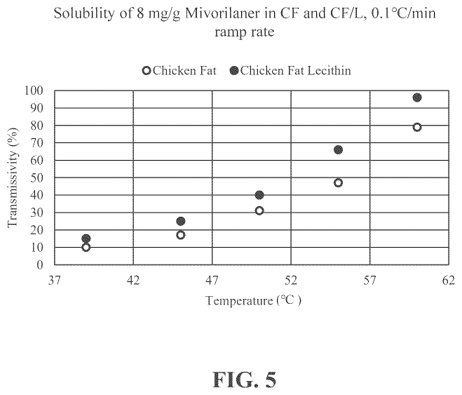

Referring now to FIG. 5, the resulting mivorilaner solubility is shown below in both chicken fat and chicken fat including 4% (w/w) lecithin. Interestingly, the presence of lecithin increased mivorilaner solubility.

The solubility of mivorilaner in a number of carriers was also measured under isothermal conditions. In the presence of chicken fat only, visible detection of agglomerates was noted in all experiments. However, mivorilaner was demonstrated to be soluble over a wide range of concentrations and temperatures in a combination of chicken fat and lecithin.

### Summary of Liquid Admixture Experiments

Admixture experiments may be performed with active ingredients including, e.g., mivorilaner, sarolaner, lotilaner, fluralaner, afoxolaner, spinosad, pyrantel, praziquantel, fenbendazole, diethylcarbamazine, moxidectin, gabapentin and metformin in combination with different test carriers using the methodology described herein. “Apparent solubility” is a term used herein to denote that the admixture may be a true solution, i.e., an admixture in which the active ingredient completely dissolves into the carrier liquid. However, the term “apparent solubility” also can include suspensions and emulsions, for example, which resemble a true solution but are not. Exemplary results from similar tests are summarized in the following tables:

### Admixtures of Various Active Ingredients Dissolved or Suspended in Various Carriers

| | | | Time to | |
|---|---|---|---|---|
| | | Active | Achieve | |
| | | Ingredient/ | Apparent | |
| | | Carrier | Solubility | Temp |
| Active Ingredient | Carrier | (mg/g) | (hours) | (° C.) |
| Mivorilaner | Chicken | 1.1 | 1.5 | 40 |
| | Fat | | | |
| Mivorilaner | Chicken | 2.0 | 3.0 | 40 |
| | Fat | | | |
| Mivorilaner | Chicken | 4.0 | 7.4 | 40 |
| | Fat | | | |
| Mivorilaner | Chicken | 1.1 | 0.5 | 45 |
| | Fat | | | |
| Mivorilaner | Chicken | 2.1 | 1.2 | 45 |
| | Fat | | | |
| Mivorilaner | Chicken | 4.0 | 3.3 | 45 |
| | Fat | | | |
| Mivorilaner | Chicken | 6.0 | 8.7 | 45 |
| | Fat | | | |
| Mivorilaner | Chicken | 1.0 | 0.3 | 50 |
| | Fat | | | |
| Mivorilaner | Chicken | 2.0 | 0.7 | 50 |
| | Fat | | | |
| Mivorilaner | Chicken | 4.0 | 2.0 | 50 |
| | Fat | | | |
| Mivorilaner | Chicken | 6.0 | 4.8 | 50 |
| | Fat | | | |
| Mivorilaner | Chicken | 2.0 | 4.5 | 40 |
| | Fat + | | | |
| | Lecithin | | | |
| Mivorilaner | Chicken | 4.0 | 6 | 40 |
| | Fat + | | | |
| | Lecithin | | | |
| Mivorilaner | Chicken | 2.0 | 2.5 | 45 |
| | Fat + | | | |
| | Lecithin | | | |
| Mivorilaner | Chicken | 3.9 | 1.5 | 45 |
| | Fat + | | | |
| | Lecithin | | | |
| Mivorilaner | Chicken | 5.9 | 14 | 45 |
| | Fat + | | | |
| | Lecithin | | | |
| Mivorilaner | Chicken | 2.0 | 1.0 | 50 |
| | Fat + | | | |
| | Lecithin | | | |
| Mivorilaner | Chicken | 4.0 | 1.5 | 50 |
| | Fat + | | | |
| | Lecithin | | | |
| Mivorilaner | Chicken | 6.0 | 3.0 | 50 |
| | Fat + | | | |
| | Lecithin | | | |
| Mivorilaner | Chicken | 7.9 | 8.5 | 50 |
| | Fat + | | | |
| | Lecithin | | | |
| Mivorilaner | Chicken | 2.0 | 1.5** | 40 |
| | Fat + | | | |
| | Tween 80 | | | |
| Mivorilaner | Triacetin | 2.0-15.8 | 0.5 | 30.0-30.6 |
| Mivorilaner | Triacetin | 31.1 | 1.0 | 30.9 |
| Mivorilaner | Triacetin | 60.2 | 2.5 | 33.8 |
| Mivorilaner | Triacetin | 113.7 | 13.5 | 50.1 |
| Fluralaner | Triacetin | 2.0 | Immediate | 45 |
| Fluralaner | Triacetin | 4.06 | Immediate | 45 |
| Fluralaner | Triacetin | 7.95 | Immediate | 45 |
| Fluralaner | Triacetin | 16.02 | Immediate | 45 |
| Fenbendazole | Chicken | 0.3 | 1.0 | 40.3 |
| | Fat | | | |
| Fenbendazole | Chicken | 0.5 | 2.5 | 42.4 |
| | Fat | | | |
| Fenbendazole | Chicken | 0.6 | 2.5 | 42.7 |
| | Fat | | | |
| Fenbendazole | Chicken | 0.9 | 7.0 | 49.4 |
| | Fat | | | |
| Fenbendazole | Chicken | 1.0 | 8.5 | 51.6 |
| | Fat | | | |
| Fenbendazole | Chicken | 1.2 | 13.0 | 57.8 |
| | Fat | | | |
| Fenbendazole | Chicken | 0.4 | 12.5 | 56.6 |
| | Fat + | | | |
| | Lecithin | | | |
| Fenbendazole | Chicken | 0.7 | 9 | 51.3 |
| | Fat + | | | |
| | Lecithin | | | |
| Fenbendazole | Chicken | 1.0 | 10.5 | 53.3 |
| | Fat + | | | |
| | Lecithin | | | |
| Fenbendazole | Chicken | 1.2 | 14.5 | 59.4 |
| | Fat + | | | |
| | Lecithin | | | |
| Fenbendazole | Triacetin | 0.5 | 9.5 | 44.2 |
| Fenbendazole | Triacetin | 1.0 | 20.5 | 60 |
| Praziquantel | Chicken | 2.0 | 0.5 | 39 |
| | Fat | | | |
| Praziquantel | Chicken | 4.0 | 0.5 | 39 |
| | Fat | | | |
| Praziquantel | Chicken | 8.0 | 0.5 | 39.3 |
| | Fat | | | |
| Praziquantel | Chicken | 16.0 | 0.5 | 40.2 |
| | Fat | | | |
| Praziquantel | Chicken | 31.8 | 13.5 | 58.9 |
| | Fat | | | |
| Praziquantel | Chicken | 4.0 | 14.7 | 59.7 |
| | Fat + | | | |
| | Lecithin | | | |
| Praziquantel | Chicken | 7.9 | 13.6 | 58.1 |
| | Fat + | | | |
| | Lecithin | | | |
| Praziquantel | Chicken | 32.1 | 15.5 | 60 |
| | Fat + | | | |
| | Lecithin | | | |
| Praziquantel | Triacetin | 6.0 | 0.1 | 30 |
| Praziquantel | Triacetin | 12.1 | 0.2 | 30 |
| Praziquantel | Triacetin | 23.9 | 0.5 | 30.6 |
| Praziquantel | Triacetin | 47.9 | 12.4 | 48.4 |
| Moxidectin | Chicken | 4.0 | 14 | 45 |
| | Fat + | | | |
| | Lecithin | | | |
| Moxidectin | Chicken | 4.0 | 9 | 50 |
| | Fat + | | | |
| | Lecithin | | | |
| Diethylcarbamazine | Chicken | 2.0 | <0.2 | 39 |
| | Fat | | | |
| Diethylcarbamazine | Chicken | 4.0 | <0.2 | 39 |
| | Fat | | | |
| Diethylcarbamazine | Chicken | 8.0 | <0.2 | 39 |
| | Fat | | | |
| Diethylcarbamazine | Chicken | 16.0 | <0.2 | 39 |
| | Fat | | | |
| Diethylcarbamazine | Chicken | 16.0 | <0.2 | 39 |
| | Fat + | | | |
| | Lecithin | | | |
| Diethylcarbamazine | Chicken | 31.9 | <0.2 | 39 |
| | Fat + | | | |
| | Lecithin | | | |
| Diethylcarbamazine | Chicken | 63.8 | <0.2 | 39 |
| | Fat + | | | |
| | Lecithin | | | |
| Diethylcarbamazine | Triacetin | 28 | NA | ~22 |
| Spinosad | Chicken | 4.0 | 0.3 | 39 |
| | Fat | | | |
| Spinosad | Chicken | 7.9 | 0.3 | 39 |
| | Fat | | | |
| Spinosad | Chicken | 16.0 | 0.3 | 39.1 |
| | Fat | | | |
| Spinosad | Chicken | 32.0 | 1.6 | 41.2 |
| | Fat | | | |
| Spinosad | Chicken | 24.1 | 0.3 | 39.3 |
| | Fat | | | |
| Spinosad | Chicken | 47.9 | 13 | 58 |
| | Fat | | | |
| Spinosad | Chicken | 23.9 | 11 | 54.6 |
| | Fat + | | | |
| | Lecithin | | | |
| Spinosad | Chicken | 32.3 | 5 | 45.8 |
| | Fat + | | | |
| | Lecithin | | | |
| Spinosad | Chicken | 39.6 | 14.5 | 60 |
| | Fat + | | | |
| | Lecithin | | | |
| Spinosad | Chicken | 47.9 | 15 | 59.6 |
| | Fat + | | | |
| | Lecithin | | | |
| Pyrantel | Chicken | 0.5 | 15 | 60 |
| Pamoate | Fat + | | | |
| | Lecithin | | | |
| Cyclosporin | CF/L | 4 | Mixed | 45 |
| | | | Sus/Sol | |
| Cyclosporin | CF/L | 1 | 10 | 60 |
| Cyclosporin | CF/L | 2 | 10 | 60 |
| Cyclosporin | CF/L | 4 | 10.5 | 60 |
| Cyclosporin | CF/L | 7.9 | 8.5 | 58 |
| Cyclosporin | Triacetin | 35 | | 20-22 |
| Fluralaner | Triacetin | 3 | | 20-22 |

*Some agglomeration noted even with transmissivity reported at 100%
**Precipitation observed at 9.6 h

### Admixtures of Various Active Ingredients Dissolved or Suspended in Various Carriers

| | | | Time to | |
|---|---|---|---|---|
| | | | Achieve | |
| | | Measured | Apparent | |
| Active | | Concentration | Solubility | Temp |
| Ingredient | Carrier | (mg/g) | (hours) | (° C.) |
| Sarolaner | Chicken | NA | NA | Up to 60 |
| | Fat + | | | |
| | Lecithin | | | |
| Lotilaner | Chicken | NA | NA | Up to 60 |
| | Fat | | | |
| Lotilaner | Chicken | NA | NA | Up to 60 |
| | Fat + | | | |
| | Lecithin | | | |
| Afoxolaner | Chicken | NA | NA | Up to 60 |
| | Fat | | | |
| Afoxolaner | Chicken | NA | NA | Up to 60 |
| | Fat + | | | |
| | Lecithin | | | |
| Pyrantel | Chicken | NA | NA | Up to 60 |
| pamoate | Fat | | | |
| Pyrantel | Triacetin | NA | NA | Up to 60 |
| pamoate | | | | |
| Moxidectin | Chicken | NA | NA | Up to 60 |
| | Fat | | | |
| Moxidectin | Triacetin | NA | NA | Up to 60 |
| Cyclosporin | CF | 0 | NA | Up to 60 |
| Gabapentin | CF | 0 | Insoluble | Up to 60 |
| Gabapentin | Triacetin | <1 | NA | 20-22 |
| Metformin-HCI | CF | 0 | Insoluble | Up to 60 |
| Metformin HCl | Triacetin | <1 | NA | 20-22 |

Admixtures of active ingredients in combination may also be tested. A non-limiting example is the combination of mivorilaner, moxidectin and fenbendazole in the carrier CF/L at (45° C.) is shown in the table, below:

| | | | Apparently |
|---|---|---|---|
| Mivorilaner | Moxidectin | Fenbendazole | dissolved after |
| (mg/g) | (mg/g) | (mg/g) | 16 hours? |
| 2.01 | 0.11 | 0.1 | No |
| 2.64 | 0.23 | 0.4 | No |
| 3.31 | 0.37 | 0.7 | No |
| 4.02 | 0.49 | 0.98 | No |

### Example 2: Preparation of a Solution

In one non-limiting example, an admixture that is a solution may be prepared by combining about 600-700 kg of refined chicken fat and about 20-30 kg of sunflower lecithin in an agitated tank held at between 40° C. and about 60° C. The mixture will be about 95-97 wt. %, refined chicken fat and about 3-5 wt. % sunflower lecithin. An active ingredient such as mivorilaner may be added to the mixture of refined chicken fat and sunflower lecithin to create an admixture that is a solution. For example, if about 1.39 kg mivorilaner were added to approximately 700 kg of the chicken fat and lecithin mixture, an admixture with about 0.198 wt. % mivorilaner should result.

### Example 3: Creation of an Active Kibble

In one non-limiting example, an active kibble having about 60 mg of mivorilaner per kg of kibble may be made. A portion of the solution of Example 2 may be diluted with chicken fat and lecithin to form a diluted solution. The diluted solution may be held in a mixing tank at a temperature of between about 28° C. and about 55° C. A portion of the diluted solution held in the mixing tank may be used to charge a pressure vessel. A portion of the diluted solution in the pressure vessel may be sprayed on the surface of a bed of kibble while tumbling/mixing in a ribbon blender or other appropriate mixer to form a substantially homogeneous, coated feed having about 60 mg of mivorilaner per kg of kibble. One of skill in the art would expect to achieve a coefficient of variation for an active kibble made according to this example of less than 20%, particularly, less than 10%, more particularly, less than 5%.

### Example 4: Creation of an Active Kibble

In one non-limiting example, an active kibble having about 140 mg of mivorilaner per kg of kibble may be made. If the solution of Example 2 were used, there would be no need to dilute it. However, if a solution with a higher concentration were used, then a portion of the solution may be diluted with chicken fat and lecithin to form a diluted solution. The diluted solution may be held in a mixing tank at a temperature of between about 28° C. and about 55° C. A portion of the diluted solution held in the mixing tank may be used to charge a pressure vessel. A portion of the diluted solution in the pressure vessel may be sprayed on the surface of a bed of kibble while tumbling/mixing in a ribbon blender or other appropriate mixer to form a substantially homogeneous, coated feed having about 140 mg of mivorilaner per kg of kibble. One of skill in the art would expect to achieve a coefficient of variation for an active kibble made according to this example of less than 20%, particularly, less than 10%, more particularly, less than 5%.

### Example 5: Creation of an Active Kibble Using Moxidectin, Chicken Fat and Sunflower Lecithin

In one non-limiting example, an admixture that is a solution may be prepared by combining about 600-700 kg of refined chicken fat and about 20-30 kg of sunflower lecithin in an agitated tank held at between 40° C. and about 60° C. The mixture will be about 95-97 wt. %, refined chicken fat and about 3-5 wt. % sunflower lecithin. An active ingredient such as moxidectin may be added to the mixture of refined chicken fat and sunflower lecithin to create an admixture that is a solution. For example, 0.6 g of moxidectin may be added to 60 g of the prepared chicken fat and sunflower lecithin mixture. This admixture forms a stock solution. Stock solutions of moxidectin may also be prepared in other dilution ratios such as 1:500, 1:1000, etc. prior to further dilution(s) to the final desired solution admixture. The stock solution may be heated (45-50° C.) and stirred for 2 hours to create a 10 mg/g solution of moxidectin.

One of skill in the art will appreciate that, once the stock solution comprising an active ingredient is made, the stock solution may be further diluted to create a range of dosage levels by further diluting the solution with inactive ingredients. For example, the stock solution may be further diluted by adding 0.4 g of the stock solution to 1.93 kg of chicken fat including sunflower lecithin (3-5 wt. %) at 40-55° C. This admixture should result in 0.0021 mg/g of moxidectin in the final solution admixture. Of course, one of skill in the art will recognize that there are many ratios that may be used to arrive at the final desired mg of moxidectin to g of solvent.

An active kibble including about 0.147 mg of moxidectin per kg of kibble may be made. A portion of the moxidectin solution (1.93 kg) may be charged to a pressure vessel and be sprayed on the surface of a kibble (25.22 kg) while tumbling in a ribbon blender to form a substantially homogeneous, coated feed having about 0.147 mg of moxidectin per kg of kibble. Further, a dry palatant may be added as disclosed elsewhere herein. One of skill in the art will recognize that the concentration of moxidectin in the feed may be adjusted by changing either the concentration or the amount of the moxidectin solution applied. One of skill in the art would expect to achieve a coefficient of variation for an active kibble made according to this example of less than 20%, particularly, less than 10%, more particularly, less than 5%.

### Example 6: Creation of an Active Kibble Using a Suspension of Fenbendazole in Chicken Fat and Sunflower Lecithin

In one non-limiting example, an admixture that is a suspension may be prepared by combining about 600-700 kg of refined chicken fat and about 20-30 kg of sunflower lecithin in an agitated tank held at between 40° C. and about 60° C. The mixture will be about 95-97 wt. %, refined chicken fat and about 3-5 wt. % sunflower lecithin.

To prepare an admixture that is a suspension, 3.13 g of micronized fenbendazole may be added to a 1.93 kg the prepared chicken fat and sunflower lecithin mixture. The desired dose may be used to determine the amount of chicken fat and sunflower lecithin to be used. This mixture may be heated (40-50° C.) and vigorously mixed (using a chopper blade or homogenizer) for approximately 10-20 minutes (based on the efficiency of the mixing system) to create a suspension admixture including fenbendazole at a target concentration of 1.62 mg/g.

An active kibble having about 115 mg of fenbendazole per kg of kibble may be made. 1.93 kg of the suspension admixture of fenbendazole as described above may be charged to a pressure vessel and be sprayed on the surface of 25.22 kg kibble while tumbling in a ribbon blender to form a substantially homogeneous, coated feed having about 115 mg of fenbendazole per kg of kibble. This dose may be adjusted by modifying the amount or concentration of the suspension admixture and/or the amount of kibble. One of skill in the art would expect to achieve a coefficient of variation for an active kibble made according to this example of less than 20%, particularly, less than 10%, more particularly, less than 5%.

### Example 7: Creation of an Active Kibble Using a Solid Diluent/Carrier Admixture with Fenbendazole

In one non-limiting example, an admixture that is a solid diluent/carrier admixture may be made. For example, 1:100 solid carrier admixture may be prepared by adding 3.13 g of micronized fenbendazole to 313.7 g green pea flour. One of skill in the art will appreciate that other dilution rates may be varied and that other solid diluents/carriers are possible, including various grain flours or fibers or other common feed ingredients. The admixture of fenbendazole/green pea flour may be tumble mixed for at least about 100 revolutions (or a sufficient number of revolutions to achieve adequate mixing based upon the mixing system selected to efficiently mix the solid diluent/carrier admixture) to prepare a visually homogeneous powder.

An active kibble having about 115 mg of fenbendazole per kg of kibble may be made. In this example, a coating solution of chicken fat having 4 wt. % sunflower lecithin may be prepared by mixing the chicken fat and sunflower lecithin at 40-55° C. to create the coating solution. The coating solution may be charged into a pressure vessel and sprayed onto the surface of the kibble while tumbling in a ribbon blender to form a substantially homogenous, coated kibble that contains no active ingredients at this stage. The active kibble may then be prepared by sifting the fenbendazole/green pea flour solid diluent/carrier admixture onto the surface of the kibble while tumbling in a ribbon blender to form a visually, substantially homogeneous, coated feed having the desired mg of fenbendazole per kg of kibble. The amount of active ingredient per portion of kibble is a function of the amount of active in the solid admixture and the percent inclusion (or inclusion rate) of the solid admixture to the kibble. Accordingly, the amount of fenbendazole/green pea flour solid diluent/carrier admixture to be used can be determined based on the percent inclusion (or inclusion rate) of the active ingredient in the solid admixture and the desired dosage for the finished kibble. For example, to achieve a dosage of approximately 115 mg of fenbendazole per kg of kibble, about 313 g of 1:100 fenbendazole/green pea flour solid diluent/carrier admixture may be sifted onto the surface of about 27 kg of the coated kibble while tumbling in a ribbon blender to form a visually, substantially homogeneous, coated feed.

One of skill in the art would expect to achieve a coefficient of variation for an active kibble made according to this example of less than 30%, particularly, less than 20%, more particularly, less than 10%. One of skill in the art would also appreciate that coating kibble with admixtures that include proteins, carbohydrates and fat also affects the nutritional content of the final product. Thus, a dilution rate for the active ingredient in the solid diluent must be chosen to optimize the desired nutritional content of the finished product as well as the desired dosage of the active ingredient in the final product.

### Example 8: Creation of a Combination Active Kibble Using an Admixture Comprising a Solution of Mivorilaner and Moxidectin and Fenbendazole in a Suspension

An admixture including multiple active ingredients may be made. In this example, a solution of mivorilaner in chicken fat including sunflower lecithin (4 wt. %) may be made as previously described in Example 2 at a concentration of about 1.62 mg mivorilaner per g of admixture.

An aliquot of 1:100 solution of moxidectin in chicken fat including sunflower lecithin (4 wt. %) as previously described in Example 5 may be added to the mivorilaner solution to provide a target concentration of moxidectin of about 0.0022 mg/g.

A suspension with fenbendazole may be prepared according to Example 6 at a concentration of about 1.62 mg of fenbendazole per g of the final combination solution/suspension admixture. The micronized fenbendazole suspension may be added to the resulting solution of mivorilaner and moxidectin to create an admixture with all three active ingredients. One of skill in the art will appreciate that a suspension of fenbendazole may be produced by adding micronized fenbendazole directly to the solution of other active ingredients, e.g., the solution of moxidectin and mivorilaner.

An active kibble comprising a combination of active ingredients may be made using an admixture, such as the admixture of mivorilaner, moxidectin and fenbendazole described above. 1.93 kg of an admixture including 1.62 mg/g of mivorilaner and 0.0022 mg/g of moxidectin in solution and 1.62 mg/g of fenbendazole in suspension may be prepared at about 40-55° C. The admixture may be charged into a pressure vessel and sprayed onto the surface of the kibble while tumbling in a ribbon blender to form a substantially homogenous, coated feed having about 115 mg of mivorilaner per kg of kibble, about 0.158 mg of moxidectin per kg of kibble, and about 115 mg of fenbendazole per kg of kibble.

### Example 9: Creation of a Combination Active Kibble Including Mivorilaner and a Solid Diluent/Carrier Admixture of Moxidectin and Fenbendazole

A solid diluent/carrier admixture including multiple active ingredients may be made. A 1:1000 solid carrier admixture (or other desired ratios) may be prepared by adding moxidectin (0.2 g) to green pea flour (200 g). Other solid diluents/carries may be various grain flours or fibers or other common feed ingredients that could serve as a carrier. The 1:1000 moxidectin/green pea flour may be tumble mixed for 100 revolutions (or a sufficient number of revolutions to achieve adequate mixing based upon the mixing system selected to efficiently mix the solid diluent/carrier admixture) to prepare a visually homogeneous powder. An aliquot of the moxidectin/pea flour admixture (1.9 g) may be weighed and added to an additional portion of pea flour (97 g). Fenbendazole (micronized) may be weighed (1.15 g) and added to the moxidectin/pea flour admixture. The final combination solid diluent/carrier admixture may be tumble mixed for 100 revolutions (or a sufficient number of revolutions to achieve adequate mixing based upon the mixing system selected to efficiently mix the solid diluent/carrier admixture) to prepare a visually homogeneous, combination admixture powder with target active ingredient concentrations of moxidectin of about 0.019 mg per g of admixture and fenbendazole of about 11.5 mg per g of admixture.

A combination active kibble including multiple active ingredients may be made. In this example, an active kibble feed including mivorilaner (115 mg of mivorilaner per kg of feed) may be prepared as previously shown in Example 4. The mivorilaner active kibble (10 kg) may be added to a tumble coater. A combination solid diluent/carrier admixture including the active ingredients moxidectin and fenbendazole in a green pea flour carrier may be prepared as described above. The combination solid diluent/carrier admixture (100 g) may be sifted onto the surface of the mivorilaner active kibble and mixed in the tumble coater over four-portion additions of the admixture to form a substantially homogenous, coated feed. Alternatively, the admixture may be added to the mivorilaner active feed in a continuous manner while mixing in the coater/mixer system. The total amount of combination solid diluent/carrier admixture added to create the final active feed may be about 0.5-2% inclusion rate of the final feed. In this example, the active ingredient final feed concentrations were about 115 mg mivorilaner per kg of feed, 115 mg of fenbendazole per kg of feed, and 0.184 mg of moxidectin per kg of feed.

### Example 10: Creation of an Active Kibble Using a Suspension of Gabapentin in Chicken Fat and Sunflower Lecithin

In manufacturing, the volumes are only limited by the size of the equipment. Therapeutic doses of gabapentin may be in a range of, for example, about 60-5,000 mg of gabapentin per kg of kibble or, more preferably about 300-3,000 mg of gabapentin per kg of kibble. An example is shown here for a particular dose of gabapentin, but one of skill in the art will understand that different dosages are possible.

In one non-limiting example, an admixture that is a suspension may be prepared by combining about 600-700 kg of refined chicken fat and about 20-30 kg of sunflower lecithin in an agitated tank held at between 40° C. and about 60° C. The mixture will be about 95-97 wt. %, refined chicken fat and about 3-5 wt. % sunflower lecithin.

To prepare an admixture that is a suspension, 31.3 g of micronized gabapentin may be added to a 1.93 kg the prepared chicken fat and sunflower lecithin mixture. The desired dose may be used to determine the amount of chicken fat and sunflower lecithin to be used. This mixture may be heated (40-50° C.) and vigorously mixed (using a chopper blade or homogenizer) for approximately 10-20 minutes (based on the efficiency of the mixing system) to create a suspension admixture including gabapentin at a target concentration of 15.9 mg/g.

An active kibble having about 1115 mg of gabapentin per kg of kibble may be made. 1.93 kg of the suspension admixture of gabapentin as described above may be charged to a pressure vessel and be sprayed on the surface of 25.22 kg kibble while tumbling in a ribbon blender to form a substantially homogeneous, coated feed having about 1115 mg of gabapentin per kg of kibble. This dose may be adjusted by modifying the amount or concentration of the suspension admixture and/or the amount of kibble. One of skill in the art would expect to achieve a coefficient of variation for an active kibble made according to this example of less than 20%, particularly, less than 10%, more particularly, less than 5%.

### Example 11: Creation of an Active Kibble Using a Solid Diluent/Carrier Admixture with Metformin

In manufacturing, the volumes are only limited by the size of the equipment. Therapeutic doses of metformin may be in a range of, for example, about 250-10,000 mg of metformin per kg of kibble, or more preferably about 1,500-5,000 mg. An example is shown here for a particular dose of metformin, but one of skill in the art will understand that different dosages are possible.

In one non-limiting example, an admixture that is a solid diluent/carrier admixture may be made. For example, a 25:100 solid carrier admixture may be prepared by adding 78.25 g of micronized metformin to 313.7 g green pea flour. One of skill in the art will appreciate that other dilution rates may be varied and that other solid diluents/carriers are possible, including various grain flours or fibers or other common feed ingredients. The admixture of metformin/green pea flour may be tumble mixed for at least about 100 revolutions (or a sufficient number of revolutions to achieve adequate mixing based upon the mixing system selected to efficiently mix the solid diluent/carrier admixture) to prepare a visually homogeneous powder.

An active kibble having about 2875 mg of metformin per kg of kibble may be made. In this example, a coating solution of chicken fat having 4 wt. % sunflower lecithin may be prepared by mixing the chicken fat and sunflower lecithin at 40-55° C. to create the coating solution. The coating solution may be charged into a pressure vessel and sprayed onto the surface of the kibble while tumbling in a ribbon blender to form a substantially homogenous, coated kibble that contains no active ingredients at this stage. The active kibble may then be prepared by sifting the metformin/green pea flour solid diluent/carrier admixture onto the surface of the kibble while tumbling in a ribbon blender to form a visually, substantially homogeneous, coated feed having the desired mg of metformin per kg of kibble. The amount of active ingredient per portion of kibble is a function of the amount of active in the solid admixture and the percent inclusion (or inclusion rate) of the solid admixture to the kibble.

Accordingly, the amount of metformin/green pea flour solid diluent/carrier admixture to be used can be determined based on the percent inclusion (or inclusion rate) of the active ingredient in the solid admixture and the desired dosage for the finished kibble. For example, to achieve a dosage of approximately 2875 mg of metformin per kg of kibble, about 313 g of 25:100 metformin/green pea flour solid diluent/carrier admixture may be sifted onto the surface of about 27 kg of the coated kibble while tumbling in a ribbon blender to form a visually, substantially homogeneous, coated feed.

One of skill in the art would expect to achieve a coefficient of variation for an active kibble made according to this example of less than 30%, particularly, less than 20%, more particularly, less than 10%. One of skill in the art would also appreciate that coating kibble with admixtures that include proteins, carbohydrates and fat also affects the nutritional content of the final product. Thus, a dilution rate for the active ingredient in the solid diluent must be chosen to optimize the desired nutritional content of the finished product as well as the desired dosage of the active ingredient in the final product.

### Example 12: Creation of an Active Kibble Using a Solution Comprising Cyclosporin

In manufacturing, the volumes are only limited by the size of the equipment. Therapeutic doses of cyclosporin may be in a range of, for example, about 50-3000 mg of cyclosporin per kg of kibble or, more preferably, about 300-2000 of cyclosporin per kg of kibble. An example is shown here for a particular dose of cyclosporin but one of skill in the art will understand that different dosages are possible.

In one non-limiting example, an admixture that is a solution may be prepared by combining about 600-700 kg of refined chicken fat and about 20-30 kg of sunflower lecithin in an agitated tank held at between 40° C. and about 60° C. The mixture will be about 95-97 wt. %, refined chicken fat and about 3-5 wt. % sunflower lecithin. An active ingredient such as cyclosporin may be added to the mixture of refined chicken fat and sunflower lecithin to create an admixture that is a solution.

For example, to prepare an admixture that is a solution, 15.65 g of cyclosporin may be added to 1.93 kg of the prepared chicken fat and sunflower lecithin. This mixture may be heated to about 40-50° C. and vigorously mixed for approximately 10-20 minutes to create a solution admixture including cyclosporin at a target concentration of about 8.0 mg/g.

An active kibble including about 575 mg of cyclosporin per kg of kibble may be made. A portion of the cyclosporin solution (1.93 kg) may be charged to a pressure vessel and be sprayed on the surface of a kibble (25.22 kg) while tumbling in a ribbon blender to form a substantially homogeneous, coated feed having about 575 mg of cyclosporin per kg of kibble. Further, a dry palatant may be added as disclosed elsewhere herein. One of skill in the art will recognize that the concentration of cyclosporin in the feed may be adjusted by changing either the concentration or the amount of the cyclosporin solution applied. One of skill in the art would expect to achieve a coefficient of variation for an active kibble made according to this example of less than 20%, particularly, less than 10%, more particularly, less than 5%.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A method of making an animal feed, comprising:
a) mixing at least one edible carrier to form a mixture, wherein the mixture optionally comprises at least one surface-active agent;
b) maintaining the mixture at a temperature greater than 40.0° C.;
c) adding at least one active ingredient to the mixture to form an admixture; and
d) maintaining the admixture at a temperature of greater than 40.0° C.

2. The method according to claim 1, further comprising:
e) providing a dry or semi-dry kibble; and
f) applying at least a portion of the admixture to at least a portion of the kibble to form an active kibble.

3. The method according to claim 2, further comprising:
g) adding at least one additive to the active kibble.

4. The method according to claim 3, further comprising:
h) tumbling the active kibble to create a substantially homogenous batch of active kibble.

5. The method according to claim 4, further comprising repeating one or more of steps f), g) and/or h).

6. The method according to claim 1, wherein the edible carrier is a synthetic or naturally occurring edible fat or oil.

7. The method according to claim 6, wherein the edible carrier is the naturally occurring edible fat or oil and is an animal fat selected from the group consisting of chicken fat, turkey fat, duck fat, fish oil, tallow, sheep fat, pork fat, or a vegetable fat or oil.

8. The method according to claim 1, wherein the active ingredient is one or more compounds selected from the group consisting of: an anthelmintic, an acaricide, a miticide, an insecticide, an analgesic, an antibiotic and a metabolic regulator.

9. The method according to claim 1, wherein the active ingredient is mivorilaner.

10. The method according to claim 1, wherein the surface-active agent is lecithin.

11. The method according to claim 1, wherein the edible carrier is chicken fat.

12. A formulation, comprising:
an admixture maintained at a temperature between about 40.0° C. and about 65.0° C., the admixture comprising:
an edible carrier;
an optional surface-active agent; and
an active ingredient.

13. The formulation of claim 12, wherein at least one of the surface-active agents is lecithin.

14. The formulation of claim 12, wherein the edible carrier is a chicken fat.

15. The formulation according to claim 12, wherein the active ingredient is mivorilaner.

16. A dry carrier admixture, comprising:
a dry edible carrier;
a solid dispersing agent; and
an active ingredient, wherein the dry carrier is mixed with the active ingredient and with the surface-active agent to form a dry admixture; and
wherein the dry admixture is configured for at least partially coating the surface of a kibble for feeding to an animal.

17. The admixture according to claim 16, wherein the dry carrier is selected from the group consisting of: pea flour, soy flour, rice flour and wheat flour.

18. The admixture according to claim 16, wherein the optional solid dispersing agent is selected from the group consisting of: polyvinyl pyrrolidone, sodium hexametaphosphate, lignosulfonates, and Na-EDTA.

19. The admixture according to claim 16, wherein the active ingredient is fenbendazole.

20. The admixture according to claim 16, wherein the active ingredient is metformin.

21. The admixture according to claim 16, further comprising a plurality of kibbles, wherein the dry carrier admixture at least partially coats the surface of the kibbles.
